(19) United States
(12) Patent Application Publication
(10) Pub. No.: US 2022/0362329 A1
(43) Pub. Date: Nov. 17, 2022

(54) TREATING ACUTE LIVER DISEASE WITH TLR-MIK INIHIBITORS

(71) Applicant: Yeda Research and Development Co. Ltd., Rehovot (IL)

(72) Inventors: Eran ELINAV, Mazkeret Batya (IL); Ido AMIT, Rehovot (IL); Aleksandra Anna KOLODZIEJCZYK, Rehovot (IL)

(73) Assignee: Yeda Research and Development Co. Ltd., Rehovot (IL)

(21) Appl. No.: 17/875,551

(22) Filed: Jul. 28, 2022

Related U.S. Application Data
(63) Continuation of application No. PCT/IL2021/050097, filed on Jan. 28, 2021.
(60) Provisional application No. 63/061,934, filed on Aug. 6, 2020.

(30) Foreign Application Priority Data
Jan. 30, 2020 (IL) 272388

(51) Int. Cl. A61K 38/14; A61P 1/16; A61K 31/43; A61K 31/7036; A61K 31/4164

(52) U.S. Cl. CPC A61K 38/14; A61P 1/16; A61K 31/43; A61K 31/7036; A61K 31/4164; A61K 38/00

(57) ABSTRACT

Methods of treating acute liver disease acute liver diseases are provided. Accordingly, there is provided a method of treating acute liver disease in a subject in need thereof comprising administering to the subject a therapeutically effective amount of an agent capable of binding a component of a TLR-MYC signaling pathway selected from the group consisting of MYC, MYD88, TRIF and p38 and inhibiting expression and/or activity of the component.

### RELATED APPLICATIONS

This application is a Continuation of PCT Patent Application No. PCT/IL2021/050097 having International filing date of Jan. 28, 2021, which claims the benefit of priority of Israel Patent Application No. 272388 filed on Jan. 30, 2020 and under 35 USC § 119(e) of U.S. Provisional Patent Application 63/061,934 filed on Aug. 6, 2020. The contents of the above applications are all incorporated by reference as if fully set forth herein in their entirety.

### SEQUENCE LISTING STATEMENT

The XML file, entitled 93233SequenceListing.xml, created on Jul. 28, 2022, comprising 23,400 bytes, submitted concurrently with the filing of this application is incorporated herein by reference.

### FIELD AND BACKGROUND OF THE INVENTION

The present invention, in some embodiments thereof, relates to methods of treating acute liver disease.

Acute liver failure (ALF) constitutes a medical emergency, in which a fulminant functional hepatic insufficiency leads to a rapid clinical deterioration and a high mortality rate in the absence of liver transplantation¹. Multiple infectious, immune, metabolic, and neoplastic diseases may manifest as ALF. Of these, acetaminophen (APAP) overdose is a leading cause for ALF in developed countries, and is caused by saturation of the normal glucuronidation and sulfation-dependent detoxification pathways, leading to an alternative hepatocyte cytochrome P450-mediated drug metabolism into N-acetyl-p-benzoquinone imine (NAPQI), driving oxidative stress and hepatocyte necrosis^{2,3}.

While ALF, including APAP induced ALF, is extensively studied, a comprehensive high-resolution cellular characterization of the events leading to liver insufficiency remains unexplored, resulting in a lack of sufficient global understanding of the molecular basis of ALF and identification of ALF therapeutic targets. As such, ALF treatment remains limited and mostly supportive. Currently, beside liver transplantation, intravenous N-acetylcysteine constitutes the APAP-induced ALF treatment, by replenishing glutathione reserves depleted in APAP detoxification. This intervention is only very partially effective and accompanied by adverse effects including anaphylactic reaction in as many as 15% of the cases⁵⁴. Even less therapeutic options are available in other ALF entities.

Additional background art includes:

Nancy Rolando et al. (1996) Semin Liver Dis, 6(4): 389-402;

Itazawa, T., et al (2010) J Gastroenterol Hepatol, 25, 1009-1012;

Shah N, et al. (2013) Liver Transpl; 19: 751-761;

Park S H et al. (2017) Exp Mol Med.; 49(11):e392;

Fisher J E et al. (2013) J Surg Res.; 180:147-55;

Park S H et al. (2015) J Immunol.; 194(3):1122-30;

Min Shi et al. Mediators of Inflammation Volume 2019, Article ID 7634761;

You-Li Yao et al. (2017) Toxicology Letters 281;

Yan-Ling Wu et al. (2009) Phytomedicine: international journal of phytotherapy and phytopharmacology, 17(6):475-9;

Wang W et al. (2018) Biomed Pharmacother.; 103:1137-1145;

Manon Fortier et al. (2019) Scientific Reports volume 9, Article number: 14614;

Jun Zhang et al. (2017) Cell Death & Disease 8(6);

Kang Zheng et al. (2017) Genes (Basel); 8(4): 123;

Nevzorova Y. A., et al. (2013) Biochim. Biophys. Acta.; 1832:1765-1775;

Ting Liu et al., Mediators of Inflammation Volume 2015, Article ID 276850;

International Patent Application Publication Nos. WO2011008640, WO2008050144 and WO2016172112;

US Patent Application Publication Nos. US20090181910, US20100016262 and US20130324471;

U.S. Pat. No. 9,187,559;

EP Patent No. EP2605775 and EP2442803; and

Canadian Patent No. CA2851663.

### SUMMARY OF THE INVENTION

According to an aspect of some embodiments of the present invention there is provided an agent capable of binding a component of a TLR-MYC signaling pathway selected from the group consisting of MYC, MYD88, TRIF and p38 and inhibiting expression and/or activity of the component, wherein when the component comprises p38 the agent inhibits activity and not expression of the p38, for use in treating acute liver disease, wherein said acute liver disease is not caused by a hepatitis C virus.

According to an aspect of some embodiments of the present invention there is provided an agent capable of binding a component of a TLR-MYC signaling pathway selected from the group consisting of MYC, MYD88 and TRIF and inhibiting expression and/or activity of the component, for use in treating acute liver disease.

According to an aspect of some embodiments of the present invention there is provided a method of treating acute liver disease in a subject in need thereof, wherein said acute liver disease is not caused by a hepatitis C virus, the method comprising administering to the subject a therapeutically effective amount of an agent capable of binding a component of a TLR-MYC signaling pathway selected from the group consisting of MYC, MYD88, TRIF and p38 and inhibiting expression and/or activity of the component, wherein when the component comprises p38 the agent inhibits activity and not expression of the p38, thereby treating the acute liver disease in the subject.

According to an aspect of some embodiments of the present invention there is provided a method of treating acute liver disease in a subject in need thereof, the method comprising administering to the subject a therapeutically effective amount of an agent capable of binding a component of a TLR-MYC signaling pathway selected from the group consisting of MYC, MYD88 and TRIF and inhibiting expression and/or activity of the component, thereby treating the acute liver disease in the subject.

According to an aspect of some embodiments of the present invention there is provided an agent capable of at least one of:

(i) binding a TLR selected from the group consisting of TLR1, TLR2, TLR3, TLR5, TLR6, TLR8 and TLR10 and inhibiting expression and/or activity of the TLR; and/or

(ii) binding at least two different TLRs and inhibiting expression and/or activity of the at least two TLRs, wherein when the TLRs are TLR7 and TLR9, the at least two is at least three, for use in treating acute liver disease.

According to an aspect of some embodiments of the present invention there is provided a method of treating acute liver disease in a subject in need thereof, the method comprising administering to the subject a therapeutically effective amount of an agent capable of at least one of:

(i) binding a TLR selected from the group consisting of TLR1, TLR2, TLR3, TLR5, TLR6, TLR8 and TLR10 and inhibiting expression and/or activity of the TLR; and/or

(ii) binding at least two different TLRs and inhibiting expression and/or activity of the at least two TLRs, wherein when the TLRs are TLR7 and TLR9, the at least two is at least three, thereby treating the acute liver disease in the subject.

According to an aspect of some embodiments of the present invention there is provided a therapeutically effective amount of antibiotic for use in treating acute liver diseases in a subject in need thereof, wherein the therapeutic effective amount inhibits TLR-MYC signaling in liver cells of the subject selected from the group consisting of stellate cells, endothelial cells and Kupffer cells.

According to an aspect of some embodiments of the present invention there is provided a method of treating acute liver disease in a subject in need thereof, the method comprising administering to the subject a therapeutically effective amount of an antibiotic, wherein the therapeutically effective amount inhibits TLR-MYC signaling in liver cells of the subject selected from the group consisting of stellate cells, endothelial cells and Kupffer cells, thereby treating the acute liver disease in the subject.

According to an aspect of some embodiments of the present invention there is provided an antibiotic for use in treating acute liver disease in a subject in need thereof, wherein the antibiotic inhibits TLR-MYC signaling in liver cells of the subject selected from the group consisting of stellate cells, endothelial cells and Kupffer cells.

According to an aspect of some embodiments of the present invention there is provided a method of treating acute liver disease in a subject in need thereof, the method comprising administering to the subject a therapeutically effective amount of an antibiotic, wherein the antibiotic inhibits TLR-MYC signaling in liver cells of the subject selected from the group consisting of stellate cells, endothelial cells and Kupffer cells, thereby treating the acute liver disease in the subject.

According to some embodiments of the invention, the use further comprising an agent capable of binding a component of a TLR-MYC signaling pathway and inhibiting expression and/or activity of the component.

According to some embodiments of the invention, the method further comprising administering to the subject a therapeutically effective amount of an agent capable of binding a component of a TLR-MYC signaling pathway and inhibiting expression and/or activity of the component.

According to some embodiments of the invention, the component is selected from the group consisting of MYC, TLR, MYD88, IRAK4, TAK1 and p38.

According to some embodiments of the invention, the TLR is not TLR4.

According to some embodiments of the invention, the component is selected from the group consisting of MYC, MYD88, IRAK4, TAK1 and p38.

According to an aspect of some embodiments of the present invention there is provided an agent capable of binding at least two components of a TLR-MYC signaling pathway and inhibiting expression and/or activity of the at least two components for use in treating acute liver disease.

According to an aspect of some embodiments of the present invention there is provided a method of treating acute liver disease in a subject in need thereof, the method comprising administering to the subject a therapeutically effective amount of an agent capable of binding at least two components of a TLR-MYC signaling pathway and inhibiting expression and/or activity of the at least two components, thereby treating the acute liver disease in the subject.

According to some embodiments of the invention, the at least two components are selected from the group consisting of MYC, TLR, MYD88, TRIF, IRAK4, TAK1 and p38.

According to some embodiments of the invention, the antibiotic is a broad spectrum antibiotic.

According to some embodiments of the invention, the antibiotic is capable of depleting a predominant portion of gut microbiome.

According to some embodiments of the invention, the agent is a small molecule.

According to some embodiments of the invention, the agent is an antibody.

According to some embodiments of the invention, the agent is an RNA silencing agent.

According to an aspect of some embodiments of the present invention there is provided a toxic agent attached to a targeting moiety for specifically targeting a cell selected from the group consisting of a stellate cell, an endothelial cell having a and a Kupffer for use in treating acute liver disease.

According to an aspect of some embodiments of the present invention there is provided a method of treating acute liver disease in a subject in a subject in need thereof, the method comprising administering to the subject a therapeutically effective amount of a toxic agent attached to a targeting moiety for specifically targeting a cell selected from the group consisting of a stellate cell, an endothelial cell and a Kupffer cell, thereby treating the acute liver disease in the subject.

According to some embodiments of the invention, the targeting moiety is an antibody.

According to some embodiments of the invention, the acute liver disease is acute liver failure.

According to some embodiments of the invention, the acute liver disease is a drug-induced acute liver disease.

According to some embodiments of the invention, the drug is acetaminophen (APAP) or thioacetamide (TAA).

According to some embodiments of the invention, the acute liver disease is caused by a virus.

According to some embodiments of the invention, the virus is Hepatitis A virus or Hepatitis B virus.

According to some embodiments of the invention, the acute liver disease is not caused by a virus.

According to some embodiments of the invention, the acute liver disease is not caused by a hepatitis C virus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEW OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D demonstrate mouse liver cell census in acute liver failure (ALF) mouse models. FIG. 1A is a schematic representation of the experiment. FIG. 1B shows UMAP visualization of cell clusters in healthy, APAP and TAA treated mice. Grey background shows all cells to aid comparison. FIG. 1C shows relative frequencies of cells in healthy and ALF mice. FIG. 1D shows Key markers used to identify cluster identity and link it to the cell type.

FIGS. 2A-2M demonstrate activation of resident cell population in ALF. FIG. 2A shows Violin plots showing normalized and scaled expression of Lrat, Col1a1 and Acta2 in stellate cells from three clusters: quiescent, fibrotic and activated (AAs). FIG. 2B shows percentage of stellate cell populations in control mice, APAP and TAA treated mice. Significance was determined using t-test. Data points from SPF samples denoted as ●, GF—▪ and ABX—♦. FIG. 2C shows balloon plots demonstrating mean normalized and scaled expression of collagens in stellate cells subpopulations. FIG. 2D is a Heatmap showing genes from extracellular matrix GO category GO:0031012 that are significantly upregulated in activated stellate cells (AAs). FIG. 2E is a Heatmap showing genes from GO category stress fiber GO:0001725 that are significantly upregulated in activated stellate cells (AAs). FIG. 2F shows Gene ontology term enrichment analysis of genes upregulated in AAs in comparison to quiescent cells. FIG. 2G shows Violin plots demonstrating expression of chemokines, cytokine and extracellular matrix regulators in stellate cell populations. FIG. 2H is a balloon plot showing normalized and scaled expression of IL6 family cytokines and their receptors in all cell types. FIG. 2I shows percentage of endothelial cell populations in control mice, APAP and TAA treated mice. Significance was determined using t-test. Data points are as in FIG. 2B. FIG. 2J shows Gene ontology term enrichment analysis of genes upregulated in AAe in comparison to sinusoidal endothelial cells. FIG. 2K shows percentage of endothelial cell populations in control mice, APAP and TAA treated mice. Significance was determined using t-test. Data points are as in FIG. 2B. FIG. 2L shows Gene ontology term enrichment analysis of genes upregulated in AAk in comparison to Kupffer cells. FIG. 2M shows balloon plots demonstrating significantly upregulated ligands in populations of stellate, endothelial and Kupffer cells and corresponding receptors and their normalized and scaled expression in all cell types.

FIGS. 3A-3K demonstrate heterogeneity of infiltrating cells in ALF. FIG. 3A is tSNE depicting two populations of neutrophils and example of genes specific for the subpopulations. FIG. 3B shows Violin plots showing normalized and scaled expression levels of chemokines, cytokines and oxidative stress response genes in neutrophil cell populations. FIG. 3C shows percentage of neutrophil populations in control mice and in APAP or TAA treated mice. Significance was determined using t-test. Data points from SPF samples denoted as ●, GF—▪ and ABX—♦. FIG. 3D is tSNE depicting two populations of Ly6C-positive monocytes and example of genes specific for the subpopulations. FIG. 3E is a bar plot showing number of differentially abundant genes between cluster of monocytes IFN and other immune cell types. FIG. 3F shows percentage of Ly6C-positive monocytes in control mice and APAP or TAA treated mice. Significance was determined using t-test. Data points are as in FIG. 3C. FIG. 3G shows percentage of monocytes IFN in control mice and APAP or TAA treated mice. Data points are as in FIG. 3C. FIG. 3H shows Gene ontology term enrichment analysis of genes upregulated in monocytes IFN in comparison to Ly6C-positive monocytes. FIG. 3I shows transcription factor binding sites enriched in the promoters of genes upregulated in monocytes IFN in comparison to Ly6C-positive monocytes. FIG. 3J shows diffusion maps explaining heterogeneity within Ly6C-positive monocytes. FIG. 3K shows Diffusion maps depicting expression of genes that change during homing process.

FIGS. 4A-4J demonstrate that common activation signature of resident cells is regulated by MYC. FIG. 4A is a Venn diagram showing overlap between sets of upregulated genes in Kupffer, stellate and endothelial cells. FIG. 4B shows transcription factor binding sites enriched in the promoters of 82-gene common activation signature. FIG. 4C shows FACS analysis of percentage of Ly6C-positive monocytes within all immune cells in the presence and absence of MYC inhibitor (MYCi) in APAP liver failure model. Significance was determined using t-test. FIGS. 4D-4E shows activity of hepatic enzyme alanine transaminase (ALT) and aspartate aminotransferase (AST) in serum of mice from APAP liver failure model in the presence and absence of MYCi. Significance was determined using t-test. FIGS. 4F-4G show representative images and histology score of H&E stained liver sections from APAP liver failure model in the presence and absence of MYCi. Significance was determined using Wilcoxon rank sum test. FIG. 4I shows Violin plots showing normalized and scaled expression of Ccl2 and Cxcl2 in Kupffer cells. FIG. 4J is a balloon-plot showing normalized and scaled expression of 82-gene common activation signature in presence and absence of MYC inhibition in stellate, endothelial and Kupffer cells.

FIGS. 5A-5J demonstrate that the microbiome modulates response to ALF via MYC and TLR. FIG. 5A shows FACS analysis of percentage of Ly6C-positive monocytes within all immune cells in germ free and SPF mice. Significance was determined using t-test. FIG. 5B shows Violin plots demonstrating normalized and scaled expression of genes differentially expressed between healthy germ free and SPF mice: Cxcl14 in stellate cells and Trf in cholangiocytes. FIG. 5C shows Violin plots demonstrating normalized and scaled expression of examples of common genes in three activated cells types that differentially expressed between germ free and SPF conditions. FIG. 5D shows expression of 82-gene common activation signature in GF, ABX and SPF mice in activated resident cell types. Significance was calculated using tailed paired Wilcoxon rank-sum test. FIG. 4E-4G show Violin plots demonstrating normalized and scaled expression of Ccl2, Mt2, Acta2 and Csf1 in activated stellate cells (FIG. 5E), activated endothelial cells (FIG. 5F) and activated Kupffer cells (FIG. 5G) in wild type mice, in the presence of MYCi and in MyD88 Trif KO mice. FIG. 5H shows box-plots demonstrating pseudobulk tpm counts of 82-gene common activation signature in wild type mice, in the presence of MYCi and in MyD88 Trif KO mice. FIGS. 5I-5J shows bar-plots demonstrating infiltration of Ly6C positive monocytes (FIG. 5I) and neutrophils (FIG. 5J) in the presence and absence of MYCi and in MyD88 Trif KO mice.

FIGS. 6A-6G demonstrate the ALF model used and characterization of cell types. FIG. 6A shows activity of hepatic enzymes aspartate transaminase (AST) and alanine transaminase (ALT) in serum of mice injected with APAP or TAA. Significance was determined using t-test. FIG. 6B shows FACS analysis of retinoid fluorescence positive cells. FIG. 6C shows activity of hepatic enzymes aspartate transaminase (AST) and alanine transaminase (ALT) in serum of germ free (GF), antibiotics treated (ABX) and specific pathogen free (SPF) mice injected with APAP or TAA. FIG. 6D shows box-plots demonstrating expression of MHCII in each cell type. FIG. 6E is a box-plot showing number of genes expressed in each cell type. FIG. 6F is a box-plot showing number of UMIs detected in each cell type, which corresponds to the number of detected unique transcripts. FIG. 6G is a boxplot showing percent of reads mapping to the mitochondrial genome in each cell type.

FIGS. 7A-7E demonstrate changes in cell abundance in ALF and differences between APAP and TAA models. FIG. 7A shows percentage of cell populations in control mice and in APAP or TAA treated mice. Significance was determined using t-test. Data points from SPF samples denoted as ●, GF—▪ and ABX—♦. FIG. 7B is a Heatmap showing differentially expressed genes in AAs cells between APAP and TAA treated mice. FIG. 7C shows Violin plots demonstrating normalized and scaled expression of example chemokines, cytokines and extracellular matrix modifiers upregulated in activated endothelial cells. FIG. 7D is a Heatmap showing differentially expressed genes in AAe cells between APAP and TAA treated mice. FIG. 7E shows Violin plots demonstrating normalized and scaled expression of example chemokines upregulated in activated Kupffer cells.

FIG. 8 demonstrates chemo-attraction in ALF. Shown in a box-plot demonstrating normalized and scaled expression of Ccl2 receptor, Ccr2 in all cell types.

FIGS. 9A-9C demonstrate cellular states upon MYC inhibition. FIG. 9A shows Violin plots demonstrating mild upregulation of expression of Myc in activated cells. FIG. 9B shows FACS gating strategy to identify Ly6C positive monocytes. FIG. 9C shows UMAP demonstrating distribution of cell clusters in healthy and in APAP or TAA treated mice, in the presence and absence of MYCi.

FIGS. 10A-10H demonstrate that effect of MYC inhibition on gene expression. FIGS. 10A-10B shows Gene ontology term enrichment analysis of genes differentially expressed in healthy mice and healthy mice treated with MYCi in stellate cells and in Kupffer cells. FIG. 10C shows Volcano-plots demonstrating differentially abundant genes healthy mice and healthy mice treated with MYCi. FIGS. 10D-E show bar-plots demonstrating infiltration of Ly6C positive monocytes and neutrophils in the presence and absence of MYCi. FIG. 10F is a Violin plot showing normalized and scaled expression of Cdkn1a in three activated cells types. FIG. 10G shows box-plots demonstrating expression of 82-gene signature in healthy mice, mice treated with APAP or TAA and mice treated with APAP and MYCi. FIG. 10H shows Gene ontology term enrichment analysis of genes differentially expressed in APAP or TAA treated mice with and without MYC inhibitor in stellate, endothelial and Kupffer cells.

FIGS. 11A-11C demonstrate the microbiome in ALF. FIG. 11A shows PCA of 16S microbiome ASV abundance data in the small intestine and in the colon of control mice or mice treated with APAP (intraperitoneal injection). FIG. 11B shows Volcano-plots demonstrating differential abundance analysis fold change and Benjamini-Hochberg adjusted p-values obtained with Wilcoxon test. FIG. 11C shows Alpha diversity metrics. Significance was determined using t-test.

FIGS. 12A-12D demonstrate microbiome effect on cellular responses in ALF. FIGS. 12A-C show Heatmaps demonstrating differentially expressed genes between activated cells in GF APAP-induced and SPF APAP-induced mice. The Heatmaps also show expression of these genes in quiescent cells in healthy mice and in activated cells in mice treated with APAP. FIG. 12D is a Violin plot showing normalized and scaled expression of TLP2 coding gene Map3k8 in activated resident populations in presence and absence of MYC inhibition.

FIGS. 13A-13C demonstrate the cellular response to APAP in MyD88 Trif KO mice. FIG. 13A shows relative frequencies of cells in healthy and mice with ALF in the presence and absence of MYCi; and in MyD88 Trif-dKO mice. FIG. 13B shows Gene ontology term enrichment analysis of genes in Kupffer cells significantly more abundant in wild type (top) and significantly more abundant in Myd88-Trif dKO (bottom). FIG. 13C shows Violin plots demonstrating normalized and scaled expression of key genes in activated stellate cells (top), activated endothelial cells (middle) and activated Kupffer cells (bottom) in wild type mice, in the presence of MYCi and in MyD88-Trif dKO mice.

FIGS. 14A-14E demonstrate the effect of inhibiting different components of the TLR-MYC signaling pathway on ALF. FIG. 14A shows FACS analysis of percentage of Ly6C-positive monocytes within all immune cells in the presence and absence of the indicated pathway inhibitors in APAP liver failure model. FIGS. 14B-C show activity of hepatic enzymes alanine transaminase (ALT) and aspartate aminotransferase (AST) in serum of mice from APAP liver failure model cells in the presence and absence of the indicated inhibitors. FIGS. 14D-E show representative images and histology score of H&E stained liver sections from APAP liver failure model in the presence and absence of the indicated inhibitors. Significance was determined using Wilcoxon rank sum test.

FIG. 15 is a schematic representation of the novel TLR-MYC signaling pathway.

FIGS. 16A-16F demonstrate cellular states upon MYC inhibition. FIG. 16A is a density plot of permutation analysis of number of MYC binding sites in randomly chosen 77 genes (black) in comparison to 77-gene signature (green). FIG. 16B shows violin plots showing mild upregulation of expression of Myc in activated cells. FIG. 16C shows images of western blots of MYC and phospho-MYC. FIG. 16D is a graph showing quantification of the western blots shown in FIG. 16C. FIG. 16E shows the FACS gating strategy to identify Ly6C-positive monocytes. FIG. 16F shows bar-plots demonstrating relative frequencies of cells in healthy and mice with ALF in the presence and absence of MYCi.

FIG. 17 is a graph representing the quantification of MYC expression levels in healthy and ALF mice from western blots, n=20 for CTRL, n=15 for APAP and n=15 for TAA.

FIGS. 18A-18B show balloon-plots demonstrating expression of chemokines and cytokines and their receptors in ALF.

FIGS. 19A-19D demonstrate that common activation signature of resident cells is regulated by MYC. FIG. 19A shows FACS analysis of percentage of Ly6C-positive monocytes within all immune cells in the presence and absence of MYC inhibitor (MYCi) in TAA liver failure model. Significance was determined using t-test. FIG. 19B shows activity of hepatic enzyme alanine transaminase (ALT) and aspartate aminotransferase (AST) in serum of mice from TAA liver failure model in the presence and absence of MYCi. Significance was determined using t-test. FIGS. 19C-D show representative images and histology score of H&E stained liver sections from TAA liver failure model in the presence and absence of MYCi. Significance was determined using Wilcoxon rank sum test.

FIG. 20 shows survival curves showing survival of APAP and APAP and MYCi treated mice; n=15 per group.

FIGS. 21A-21B demonstrate TLR ligands in ALF. The Figures show heatmaps showing mean levels of 655 nm absorbance in HEK-Blue TLR and NLR reporter cell lines subtracted with absorbance of corresponding Null cell line after application of portal serum from germ free (GF), antibiotics treated (ABX) and SPF mice treated with APAP and TAA.

FIG. 22 is a violin plot showing normalised and scaled expression of TLP2 coding gene Map3k8 in activated resident populations in the presence and absence of MYC inhibition.

FIGS. 23A-23B demonstrate upregulating of MYC in human ALF. FIG. 23A shows immunohistochemistry score of MYC in human controls and ALF samples; n=5 for controls and n=7 for ALF. FIG. 23B shows representative images of MYC immunohistochemistry. Arrows indicate positive staining for MYC, black bar in histology denotes 100 μm.

### DESCRIPTION OF SPECIFIC EMBODIMENTS OF THE INVENTION

The present invention, in some embodiments thereof, relates to methods of treating acute liver disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Acute liver failure (ALF) is a fulminant life-risking disorder, driven by a variety of pharmacological, viral, metabolic and immune entities, which is characterized by a rapid hepatic destruction, multi-organ failure and mortality. ALF treatment is limited, mainly consisting of supportive care and liver transplantation.

Whilst reducing the present invention to practice, the present inventors have now uncovered that specific subsets of activated liver resident stellate, endothelial and Kupffer cells are associated with ALF. Furthermore, the present inventors elucidate a novel TLR-MYC signaling pathway driving this activation.

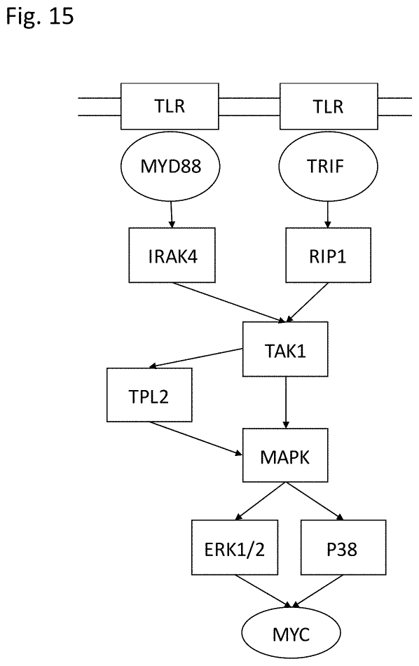

As is illustrated hereinunder and in the examples section, which follows, the present inventors show that activated liver resident stellate, endothelial and Kupffer cells drive a conserved chain of events and inter-cellular crosstalk driving ALF in two mouse models of ALF (i.e. APAP induced and TAA induced ALF) (Example 1 of the Examples section which follows). Furthermore, the inventors show that a novel TLR-MYC signaling pathway (FIG. 15) drives activation of these cells during ALF and that inhibition of components in these pathway (e.g. MYC, MYD88, IRAK4, TAK1, p38) attenuate ALF in both mouse models (Examples 2-3 of the Examples section which follows).

Consequently, specific embodiments of the present invention suggest targeting the activated stellate, endothelial and/or Kupffer cells or the TLR-MYC signaling pathway for treating acute liver diseases.

Thus, according to an aspect of the present invention, there is provided an agent capable of binding a component of a TLR-MYC signaling pathway selected from the group consisting of MYC, MYD88, TRIF and p38 and inhibiting expression and/or activity of said component, wherein when said component comprises p38 said agent inhibits activity and not expression of said p38, for use in treating acute liver disease, wherein said acute liver disease is not caused by a hepatitis C virus. According to an additional or an alternative aspect of the present invention, there is provided a method of treating acute liver disease in a subject in need thereof, wherein said acute liver disease is not caused by a hepatitis C virus, the method comprising administering to the subject a therapeutically effective amount of an agent capable of binding a component of a TLR-MYC signaling pathway selected from the group consisting of MYC, MYD88, TRIF and p38 and inhibiting expression and/or activity of said component, wherein when said component comprises p38 said agent inhibits activity and not expression of said p38, thereby treating the acute liver disease in the subject.

According to an additional or an alternative aspect of the present invention, there is provided an agent capable of binding a component of a TLR-MYC signaling pathway selected from the group consisting of MYC, MYD88 and TRIF and inhibiting expression and/or activity of said component, for use in treating acute liver disease.

According to an additional or an alternative aspect of the present invention, there is provided a method of treating acute liver disease in a subject in need thereof, the method comprising administering to the subject a therapeutically effective amount of an agent capable of binding a component of a TLR-MYC signaling pathway selected from the group consisting of MYC, MYD88 and TRIF and inhibiting expression and/or activity of said component, thereby treating the acute liver disease in the subject.

According to an additional or an alternative aspect of the present invention, there is provided an agent capable of at least one of:

(i) binding a TLR selected from the group consisting of TLR1, TLR2, TLR3, TLR5, TLR6, TLR8 and TLR10 and inhibiting expression and/or activity of said TLR; and/or

(ii) binding at least two different TLRs and inhibiting expression and/or activity of said at least two TLRs, wherein when said TLRs are TLR7 and TLR9, said at least two is at least three,

for use in treating acute liver disease.

According to an additional or an alternative aspect of the present invention, there is provided a method of treating acute liver disease in a subject in need thereof, the method comprising administering to the subject a therapeutically effective amount of an agent capable of at least one of:

(i) binding a TLR selected from the group consisting of TLR1, TLR2, TLR3, TLR5, TLR6, TLR8 and TLR10 and inhibiting expression and/or activity of said TLR; and/or

(ii) binding at least two different TLRs and inhibiting expression and/or activity of said at least two TLRs, wherein when said TLRs are TLR7 and TLR9, said at least two is at least three,

thereby treating the acute liver disease in the subject.

According to an additional or an alternative aspect of the present invention, there is provided an agent capable of binding at least two components of a TLR-MYC signaling pathway and inhibiting expression and/or activity of said at least two components for use in treating acute liver disease.

According to an additional or an alternative aspect of the present invention, there is provided a method of treating acute liver disease in a subject in need thereof, the method comprising administering to the subject a therapeutically effective amount of an agent capable of binding at least two components of a TLR-MYC signaling pathway and inhibiting expression and/or activity of said at least two components, thereby treating the acute liver disease in the subject.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition, e.g., ALF). According to specific embodiments, this term encompasses individuals who are at risk to develop the pathology.

According to specific embodiments, the subject does not present a sign or symptom of local or systemic infection, such as sepsis, cholangitis, gastrointestinal infection, pneumonia or spontaneous bacterial peritonitis. Non-limiting examples of signs or symptoms of infection include, but are not limited to, fever, hypothermia, unexplained hypotension, leukocytosis, elevation in CRP.

As used herein, the phrase “acute liver disease” refers to a liver disease of short duration, i.e., not chronic, the history of which typically does not exceed six months, wherein severe complications appear rapidly after the first signs of liver disease. The 1993 classification defines hyperacute as within 1 week, acute as 8-28 days and subacute as 4-12 weeks (Williams, et al. Lancet, 1993, 342, 273). Typically, acute liver disease is not associated with a liver fibrotic scar, in contrast to chronic liver disease. The acute liver disease may result, for example, from infectious or autoimmune processes, from mechanical or chemical (e.g. drug, toxin) injury to the liver and the like.

According to specific embodiments, the acute liver disease is drug-induced acute liver disease [e.g. acetaminophen (APAP) or thioacetamide (TAA)].

According to other specific embodiments, the acute liver disease is caused by (alternatively induced by or associated with) a virus (e.g. Hepatitis A or Hepatitis B).

According to specific embodiments, the acute liver disease is not caused (alternatively not induced by or not associated with) by a virus.

According to specific embodiments, the acute liver disease is not caused by (alternatively not induced by or not associated with) a hepatitis C virus.

According to specific embodiments, the acute liver disease is not accompanied with local or systemic infection.

Non-limiting examples of acute liver diseases include acute liver failure and acute hepatitis (e.g. acute viral hepatitis, acute autoimmune hepatitis and acute alcoholic hepatitis). Methods of assessing acute liver diseases are known in the art and include, but not limited to, clinical assessment, prothrombin time test, serum levels of liver enzymes (e.g. AST, ALT) and liver histology, as also described in details in the Examples section which follows.

According to specific embodiments, the acute liver disease is acute liver failure.

As used herein, the phrase “acute liver failure (ALF)” refers to development of sudden, severe hepatic dysfunction from an acute insult to the liver, associated with the onset of hepatic encephalopathy and coagulation abnormalities. The most widely accepted definition from the American Association for the Study of Liver Diseases (AASLD) is “evidence of coagulation abnormality, usually an international normalized ratio above 1.5, and any degree of mental alteration (encephalopathy) in a patient without preexisting liver disease and with an illness of less than 26 weeks' duration. Non-limiting examples of ALF etiologies include drug-induced liver injury [e.g. acetaminophen (APAP) or thioacetamide (TAA), antibiotics (e.g. amoxicillin-clavulanate, ciprofloxacin, nitrofurantoin, minocycline, dapsone, doxycycline, trimethoprim-sulfamethoxazole, efavirenz, didanosine, abacavir), anti-epileptics (e.g. valproic acid, phenytoin, carbamazepine), Anti-tuberculosis drugs (e.g. isoniazid, rifampin-isoniazid, pyrazinamide), propylthiouracil, amitryptiline, statins, amiodarone, methotrexate, methyldopa), NSAID (e.g. Diclofenac, ibuprofen, indomethacin, naproxen, Herbs (e.g. ma huang, kava kava, Herbalife); viral hepatitis (e.g. Hepatitis A, B, C and E, CMV, EBV, herpes virus, varicella zoster virus); pregnancy specific liver diseases (e.g. Acute fatty liver of pregnancy, HELLP syndrome, Preeclampsia-associated liver diseases); ischemic hepatitis (e.g. systemic hypotension, Budd-Chiari syndrome); autoimmune hepatitis, Wilson's disease, Mushroom poisoning.

The agents of some embodiments of the invention are capable of binding a component in a TLR-MYC signaling pathway and inhibiting expression and/or activity of the component.

As used herein the phrase “a component of a TLR-MYC signaling pathway” refers to a protein or chemical component being part of a signaling pathway that starts with activation of TLR and ends with activation of MYC, as shown in FIG. 15, which is to be considered as part of this specification. Non-limiting examples of components of the TLR-MYC signaling pathway include TLR, MYD88, TRIF, IRAK4, TAK1, RIP1, TPL2, MAPK, p38, ERK1/2 and MYC.

According to specific embodiments, the component is selected from the group consisting of MYC, TLR, MYD88, TRIF, IRAK4, TAK1 and p38.

According to specific embodiments, the component is selected from the group consisting of MYC, TLR, MYD88, IRAK4, TAK1 and p38.

According to specific embodiments, the component is selected from the group consisting of MYC, MYD88, IRAK4, TAK1 and p38.

According to specific embodiments, the component is selected from the group consisting of MYC, MYD88, TRIF and p38.

According to specific embodiments, the agent binds a single component of a TLR-MYC signaling pathway and inhibits expression and/or activity of same.

According to other specific embodiments, the agent binds at least two, at least three or at least four components of a TLR-MYC signaling pathway. Hence, according to specific embodiments, the agent binds at least MYC and TLR or at least MYD88 and TRIF. In this case, the agent may be a single agent targeting the different components or multiple agents each targeting different component(s).

According to specific embodiments the agent binds MYC and inhibits expression and/or activity of same.

As used herein, the term “MYC” also known as c-MYC, V-Myc Avian Myelocytomatosis Viral Oncogene Homolog, Class E Basic Helix-Loop-Helix Protein 39, Transcription Factor P64, BHLHe39, MRTL and MYCC, refers to the polynucleotide and expression product e.g., polypeptide of the MYC gene (Gene ID 4609). According to specific embodiments the MYC refers to the human c-MYC, such as provided in the following GeneBank Numbers NP_002458 and NM 002467.

According to specific embodiments the agent binds TLR and inhibits expression and/or activity of same.

As used herein, the term “TLR” refers to the polynucleotide and expression product e.g., polypeptide of at least one toll-like receptor gene. Toll-like receptors are a class of single-pass membrane-spanning receptors that bind to structurally conserved molecules derived from microbes. TLRs are a type of pattern recognition receptor (PRR) and their ligands (e.g. bacterial cell-surface lipopolysaccharides (LPS), lipoproteins, lipopeptides and lipoarabinomannan; proteins such as flagellin from bacterial flagella; double-stranded RNA of viruses or the unmethylated CpG motifs of bacterial and viral DNA; and certain other RNA and DNA), are known collectively as pathogen-associated molecular patterns (PAMPs). Endogenous ligands of TLRs have also been identified, including fibrinogen, heat shock proteins (HSPs), and DNA.

According to specific embodiments the TLR refers to the human TLR.

Ten TLRs have been identified in human so far, namely TLR-1 to TLR-10.

According to specific embodiments, the TLR is TLR1 (Gene ID 7096).

According to specific embodiments the TLR1 refers to the human TLR1, such as provided in the following GeneBank Numbers NM_003263 and NP_003254.

According to specific embodiments, the TLR is TLR2 (Gene ID 7097).

According to specific embodiments the TLR2 refers to the human TLR2, such as provided in the following GeneBank Numbers NM_003264, NM_001318787, NM_001318789, NM_001318790, NM_001318791, NP_001305716, NP_001305718, NP_001305719, NP_001305720 and NP_001305722.

According to specific embodiments, the TLR is TLR3 (Gene ID 7098).

According to specific embodiments the TLR3 refers to the human TLR3, such as provided in the following GeneBank Numbers NM_003265 and NP_003256.

According to specific embodiments, the TLR is TLR4 (Gene ID 7099).

According to other specific embodiments, the TLR is not TLR4.

According to specific embodiments the TLR4 refers to the human TLR4, such as provided in the following GeneBank Numbers NM_138557, NM_003266, NM_138554, NM_138556, NP_003257, NP_612564 and NP_612567.

According to specific embodiments, the TLR is TLR5 (Gene ID 7100).

According to specific embodiments the TLR5 refers to the human TLR5, such as provided in the following GeneBank Numbers NM_003268 and NP_003259.

According to specific embodiments, the TLR is TLR6 (Gene ID 10333).

According to specific embodiments the TLR6 refers to the human TLR6, such as provided in the following GeneBank Numbers NM_006068 and NP_006059.

According to specific embodiments, the TLR is TLR7 (Gene ID 51284).

According to specific embodiments the TLR7 refers to the human TLR7, such as provided in the following GeneBank Numbers NM_016562 and NP_057646.

According to specific embodiments, the TLR is TLR8 (Gene ID 51311).

According to specific embodiments the TLR8 refers to the human TLR8, such as provided in the following GeneBank Numbers NM_016610, NM_138636, NP_057694 and NP_619542.

According to specific embodiments, the TLR is TLR9 (Gene ID 54106).

According to specific embodiments the TLR9 refers to the human TLR9, such as provided in the following GeneBank Numbers NM_138688, NM_017442 and NP_059138.

According to specific embodiments, the TLR is TLR10 (Gene ID 81793).

According to specific embodiments the TLR10 refers to the human TLR10, such as provided in the following GeneBank Numbers NM_001017388, NM_001195106, NM_001195107, NM_001195108, NM_030956, NP_001017388, NP_001182035, NP_001182036, NP_001182037 and NP_112218.

According to specific embodiments, the agent binds a single type of TLR and inhibits expression and/or activity of same.

According to specific embodiments, the agent binds at least one type of TLR and inhibits expression and/or activity of same.

According to other specific embodiments, the agent binds at least two, at least three, at least four, at least five, at least six, at least seven, at least eight, at least nine or ten different TLRs, and inhibits expression and/or activity of same. In this case, the agent may be a single agent targeting the different TLRs (e.g., intracellular diabody) or multiple agents each targeting different TLR(s).

According to specific embodiments, the agent binds at least two TLRs selected from the group consisting of TLR1-TLR10.

According to specific embodiments, the agent binds at least one TLR selected from the group consisting of TLR1, TLR2, TLR3, TLR5, TLR6, TLR7, TLR8, TLR9 and TLR10 and inhibits activity of same.

According to specific embodiments, the agent binds at least one TLR selected from the group consisting of TLR1, TLR2, TLR3, TLR5, TLR6, TLR8 and TLR10 and inhibits activity of same.

According to specific embodiments, the agent binds TLR4 and at least one of TLR1, TLR2, TLR3, TLR5, TLR6, TLR7, TLR8, TLR9 and TLR10.

According to specific embodiments, the agent binds TLR7 and/or TLR9 and at least one of TLR1, TLR2, TLR3, TLR4, TLR5, TLR6, TLR8 and TLR10.

According to a specific embodiment, the agent binds TLR4 and TLR7 and/or TLR9.

According to specific embodiments the agent binds MYD88 and inhibits expression and/or activity of same.

As used herein, the term “MYD88” also known as Myeloid differentiation primary response 88, refers to the polynucleotide and expression product e.g., polypeptide of the MYD88 gene (Gene ID 4615). According to specific embodiments the MYD88 refers to the human MYD88, such as provided in the following GeneBank Numbers NM_001172566, NM_001172567, NM_001172568, NM_001172569, NM_002468, NP_001166037, NP_001166038, NP_001166039, NP_001166040 and NP_002459.

According to specific embodiments the agent binds TRIF and inhibits expression and/or activity of same.

As used herein, the term “TRIF” also known as TIR-domain-containing adapter-inducing interferon-β, refers to the polynucleotide and expression product e.g., polypeptide of the TICAM1 gene (Gene ID 148022). According to specific embodiments the TRIF refers to the human TRIF, such as provided in the following GeneBank Numbers NM_014261, NM_182919 and NP_891549.

According to specific embodiments the agent binds IRAK4 and inhibits expression and/or activity of same.

As used herein, the term “IRAK4” also known as interleukin-1 receptor-associated kinase 4, refers to the polynucleotide and expression product e.g., polypeptide of the IRAK4 gene (Gene ID 51135). According to specific embodiments the IRAK4 refers to the human IRAK4, such as provided in the following GeneBank Numbers NM_001114182, NM_001145256, NM_001145257, NM_001145258, NM_016123, NP_001107654, NP_001138728, NP_001138729, NP_001138730 and NP_057207.

According to specific embodiments the agent binds TAK1 and inhibits expression and/or activity of same.

As used herein, the term “TAK1” also known as Mitogen-activated protein kinase kinase kinase 7 (MAP3K7), refers to the polynucleotide and expression product e.g., polypeptide of the MAP3K7 gene (Gene ID 6885). According to specific embodiments the TAK1 refers to the human TAK1, such as provided in the following GeneBank Numbers NM_003188, NM_145331, NM_145332, NM_145333, NP_003179, NP_663304, NP_663305 and NP_663306.

According to specific embodiments the agent binds p38 and inhibits expression and/or activity of same.

As used herein, the term “p38” refers to the polynucleotide and expression product e.g., polypeptide of at least one p38 mitogen-activated protein kinase, a class of mitogen-activated protein kinase. According to specific embodiments, the p38 refers to the human p38. Four p38 mitogen-activated protein kinases have been identified p38α (MAPK14), p38β (MAPK11), p38γ (MAPK12/ERK6) and p38δ (MAPK13/SAP4).

According to specific embodiments, the p38 is p38α (Gene ID 1432).

According to specific embodiments the p38a refers to the human p38a, such as provided in the following GeneBank Numbers NM_001315, NM_139012, NM_139013, NM_139014, NP_001306, NP_620581, NP_620582 and NP_620583.

According to specific embodiments, the p38 is p38β (Gene ID 5600).

According to specific embodiments the p38β refers to the human p38β, such as provided in the following GeneBank Numbers NM_002751 and NP_002742.

According to specific embodiments, the p38 is p38γ (Gene ID 6300).

According to specific embodiments the p38γ refers to the human p38γ, such as provided in the following GeneBank Numbers NM_002969, NM_001303252, NP_001290181 and NP_002960.

According to specific embodiments, the p38 is p38δ (Gene ID 5603).

According to specific embodiments the p38δ refers to the human p38δ, such as provided in the following GeneBank Numbers NM_002754 and NP_002745.

Assays for testing binding are well known in the art and include, but not limited to flow cytometry, BiaCore, bio-layer interferometry Blitz® assay, HPLC.

As used herein, “inhibiting expression and/or activity” refers to a decrease of at least 5% in expression and/or biological function in the presence of the agent in comparison to same in the absence of the agent, as determined by e.g. PCR, ELISA, Western blot analysis, immunoprecipitation, flow cytometry, immuno-staining, kinase assays, binding assay and the like depending on the component tested. Alternatively or additionally, as the inhibitory agents of some embodiments of the present invention have an ameliorating effect on acute liver failure, the decrease can also be determined by liver function assessment such as prothrombin time test, serum levels of liver enzymes (e.g. AST, ALT) and liver histology, as further described in details in the Examples section which follows. Alternatively or additionally, as the inhibitory agents of some embodiments of the present invention have an effect on activated liver resident stellate, endothelial and Kupffer cells, the decrease can also be determined by cytological assays e.g. assessment of morphological, phenotypic and transcriptional changes in liver stellate, endothelial and/or Kupffer cells, as further described in details in the Examples section which follows. According to a specific embodiment, the decrease is in at least 10%, 30%, 40% or even higher say, at least 50%, at least 60%, at least 70%, at least 80%, at least 90%, at least 95% or 100%. According to specific embodiments, the decrease is at least 1.5 fold, at least 2 fold, at least 3 fold, at least 5 fold, at least 10 fold, or at least 20 fold as compared to same in the absence of the agent.

According to specific embodiments, the agent inhibits expression of the component in the TLR-MYC signaling pathway.

According to specific embodiments, the agent inhibits activity of the component in the TLR-MYC signaling pathway.

Inhibiting expression and/or activity can be can be effected at the protein level (e.g., antibodies, small molecules, inhibitory peptides, enzymes that cleave the polypeptide, aptamers and the like) but may also be effected at the genomic (e.g. homologous recombination and site specific endonucleases) and/or the transcript level using a variety of molecules which interfere with transcription and/or translation (e.g., RNA silencing agents) of a target expression product described herein.

Inhibiting expression and/or activity may be either transient or permanent.

Non-limiting examples of inhibitory agents are described in details hereinbelow.

Inhibiting at the Polypeptide Level

According to specific embodiments, the inhibiting agent is an antibody.

According to specific embodiments, the antibody specifically binds at least one epitope of a target protein described herein.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen. The antibody may be mono-specific (capable of recognizing one epitope or protein), bi-specific (capable of binding two epitopes or proteins) or multi-specific (capable of recognizing multiple epitopes or proteins).

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77].

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

The antibody may be monoclonal or polyclonal.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

It will be appreciated that for human therapy or diagnostics, humanized antibodies are preferably used. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As some of the targets described herein are localized intracellularly, the antibody or antibody fragment capable can be an intracellular antibody (also known as “intrabodies”). Intracellular antibodies are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Once antibodies are obtained, they may be tested for activity, for example via ELISA.

Another inhibitory agent which can be used along with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another inhibitory agent would be any molecule which interferes with the target protein activity (e.g., catalytic or interaction) by binding the target protein and/or cleaving the target protein. Such molecules can be a small molecule, antagonists, or inhibitory peptide.

Another inhibitory agent which can be used along with some embodiments of the invention is a molecule which prevents target activation or substrate binding.

According to a specific embodiment, the inhibitory agent is a small molecule.

According to a specific embodiment, the inhibitory agent is a peptide molecule.

It will be appreciated that a non-functional analogue of at least a catalytic or binding portion of the target can be also used as an inhibitory agent.

Inhibiting at the Nucleic Acid Level

Inhibition at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, inhibition can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., MYC) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to INHIBIT protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433; and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to down-regulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA Inhibition can be effected using an antisense polynucleotide capable of specifically hybridizing with an mRNA transcript encoding the target (e.g. MYC).

Design of antisense molecules which can be used to efficiently down-regulate a target must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jäa\ddot{s}keläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for down-regulating expression of known sequences without having to resort to undue trial and error experimentation.

Nucleic acid agents can also operate at the DNA level as summarized infra.

Inhibition can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene which results in down-regulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the gene may be in a homozygous form or in a heterozygous form.

Methods of introducing nucleic acid alterations to a gene of interest are well known in the art [see for example Menke D. Genesis (2013) 51: -618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs— Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fold domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Specific non-limiting examples of MYC inhibitors that can be used with specific embodiments include, small molecules such as KJ Pyr 9, MAD1, MAX, MNT, MXD 1-4, MGA, 10069-F4, APTO-253, MYCi975, MYCi361, Sajm589, IZCZ-3, CX-3543 (Quarlfoxin), cationic porphyrins, quindolines, platinum complexes, elipticine, 10058-f4, 10074-g5, jy-3-094, 3JC48-3, Mycro3, MI1-PD, KS0-3716; antisense molecule such as INX-3280, AVI-4216 (Resten-NG); siRNA such as SiRNA:DCR-MYC; peptides such as Omomyc, H1 peptide.

Specific non-limiting examples of TLR4 inhibitors that can be used with specific embodiments include, small molecules such as Ethyl (6R)-6-[N-(2-chloro-4-fluorophenyl)sulfamoyl]cyclohex-1-ene-1-carboxylate (TAK-242), etrasodium [(2R,3R,4R,5S,6R)-4-decoxy-5-hydroxy-6-[[(2R,3R,4R,5S,6R)-4-[(3R)-3-methoxydecoxy]-6-(methoxymethyl)-3-[[(Z)-octadec-11-enoyl]amino]-5-phosphonatooxyoxan-2-yl]oxymethyl]-3-(3-oxotetradecanoylamino)oxan-2-yl]phosphate (eritoran) which may be provided as E5564, IAXO compounds such as IAXO-101 (Methyl 6-deoxy-6-N-dimethyl-N-cyclopentylammonium-2,3-di-O-tetradecyl-α-D-glucopyranoside iodide), IAXO-102 Methyl 6-Deoxy-6-amino-2,3-di-O-tetradecyl-α-D-glucopyranoside, or IAXO-103 (N-(3,4-Bis-tetradecyloxy-benzyl)-N-cyclopentyl-N,N-dimethylammonium iodide); peptides such as STM28 (as described in Sugiyama et al (European Journal of Pharmacology 594 (2008) 152-156); antibodies such as NI-0101; phospholipids such as OxPAPC (1-palmitoyl-2-arachidonyl-sn-glycero-3-phosphorylcholine); and the compounds reviewed in Hennessy et al (2010) Nature Reviews Drug Discovery 9: 293-307, the contents of which are hereby incorporated by reference.

Specific non-limiting examples of TLR7 and/or TLR9 inhibitors that can be used with specific embodiments include, small molecules such as chloroquine, hydroxychloroquine, quinacrine and bafilomycin A, DV1079, IM03100, CPG52364, ODN2088; Oligonucleotides such as IRS954 [described in Barrat et al (Eur J Immunol 2007 37: 3582-3586) and in Barrat et al J Exp Med 2005 202: 1131-1139), the contents of which are hereby incorporated by reference], and the oligodeoxynucleotide compounds containing unmethylated CpG dinucleotides described in Yu et al (J. Med Chem, 2009, 52: 5108-5114), the contents of which are hereby incorporated by reference.

Specific non-limiting examples of MYD88 inhibitors that can be used with specific embodiments include, small molecules such as ST2825, T6167923; peptides such as NBP2-29328, Pepinh-MYD.

Specific non-limiting examples of IRAK4 inhibitors that can be used with specific embodiments include small molecules such as CA-4948 and the compounds reviewed in McElroy W T, Expert Opin Ther Pat. 2019 April; 29(4):243-259, the contents of which are hereby incorporated by reference.

Specific non-limiting examples of TAK1 inhibitors that can be used with specific embodiments include small molecules such as Takinib, NG2, 5Z-7-Oxozeaenol.

Specific non-limiting examples of p38 inhibitors that can be used with specific embodiments include small molecules such as SB203580, VX-702, AZD7624, SD 0006, VX-745, TAK-715, Pamapimod, SB239063, Skepinone, Losmapimod (GW856553X), BMS-582949, Pexmetinib (ARRY-614), UM-164.

According to an additional or an alternative aspect of the present invention, there is provided a therapeutically effective amount of antibiotic for use in treating acute liver diseases in a subject in need thereof, wherein said therapeutic effective amount inhibits TLR-MYC signaling in liver cells of said subject selected from the group consisting of stellate cells, endothelial cells and Kupffer cells.

According to an additional or an alternative aspect of the present invention, there is provided a method of treating acute liver disease in a subject in need thereof, the method comprising administering to the subject a therapeutically effective amount of an antibiotic, wherein said therapeutically effective amount inhibits TLR-MYC signaling in liver cells of said subject selected from the group consisting of stellate cells, endothelial cells and Kupffer cells, thereby treating the acute liver disease in the subject.

According to an additional or an alternative aspect of the present invention, there is provided an antibiotic for use in treating acute liver disease in a subject in need thereof, wherein said antibiotic inhibits TLR-MYC signaling in liver cells of the subject selected from the group consisting of stellate cells, endothelial cells and Kupffer cells.

According to an additional or an alternative aspect of the present invention, there is provided a method of treating acute liver disease in a subject in need thereof, the method comprising administering to the subject a therapeutically effective amount of an antibiotic, wherein said antibiotic inhibits TLR-MYC signaling in liver cells of said subject selected from the group consisting of stellate cells, endothelial cells and Kupffer cells, thereby treating the acute liver disease in the subject.

As used herein, the term “antibiotic” refers to any natural, synthetic, and semi-synthetic compound that is cytotoxic and/or cytostatic to a microorganism e.g. bacteria, fungi, viruses and/or parasites.

According to specific embodiments, the antibiotic is cytotoxic and/or cytostatic to bacteria.

According to specific embodiments, the antibiotic is cytotoxic and/or cytostatic to bacteria and fungi.

According to specific embodiments, the antibiotic is cytotoxic to the microorganism.

According to specific embodiments, the antibiotic is a broad spectrum antibiotic.

As used herein the phrase “broad spectrum antibiotic” refer to an antibiotic effective for a range of microorganisms.

According to specific embodiments, the broad spectrum antibiotic is cytotoxic and/or cytostatic for both gram-positive and gram-negative bacteria.

According to specific embodiments, the broad spectrum antibiotic is cytotoxic and/or cytostatic for gram-positive, gram-negative and anaerobic bacteria.

According to specific embodiments, the antibiotic is not Piperacillin-Tazobactam, ciprofloxacin and/or ceftriaxone.

According to specific embodiments, the antibiotic is capable of depleting a predominant portion of gut microbiome.

“A predominant portion of gut microbiome” refers herein to an antibiotic that is cytotoxic for more than 5, more than 10, more than 15, more than 20, more than 30 species present in the gut microbiome of the subject.

The antibiotic of some embodiments of the present invention inhibits TLR-MYC signaling in liver cells selected from the group consisting of stellate cells or is administered to the subject in therapeutically effective amount which inhibits TLR-MYC signaling in liver cells selected from the group consisting of stellate cells, endothelial cells and Kupffer cells.

As used herein, “inhibits TLR-MYC signaling” refers to a decrease of at least 5% in activation of the TLR-MYC signaling pathway (as shown in FIG. 15, which is to be considered as part of this specification) in liver resident stellate, endothelial and/or Kupffer cells in the presence of the antibiotic in comparison to same in the absence of the antibiotic, as determined by e.g. kinase assays, binding assays. Alternatively or additionally, determining inhibition of TLR-MYC signaling may be effected by cytological assay e.g. determining morphological, phenotypic and transcriptional changes in liver stellate, endothelial and/or Kupffer cells, as further described in details in the Examples section which follows. Alternatively or additionally, determining inhibition of TLR-MYC signaling may be effected by determining liver function such as prothrombin time test, serum levels of liver enzymes (e.g. AST, ALT) and liver histology, as further described in details in the Examples section which follows. According to a specific embodiment, the decrease is in at least 10%, 20%, 30%, 40% or even higher say, at least 50%, at least 60%, at least 70%, at least 80%, at least 90%, at least 95% or 100%. According to specific embodiments, the decrease is at least 1.5 fold, at least 2 fold, at least 3 fold, at least 5 fold, at least 10 fold, or at least 20 fold as compared to same in the absence of the agent.

According to specific embodiments, the antibiotic is administered to the subject in combination with an agent capable of binding a component of a TLR-MYC signaling pathway and inhibiting expression and/or activity of same.

According to specific embodiments, such a combined treatment has an additive effect on treatment of acute liver disease as compared to each of the agents when administered as a single therapy.

According to specific embodiments, such a combined treatment has a synergistic effect on treatment of acute liver disease as compared to each of the agents when administered as a single therapy.

As the present inventors have discovered novel populations of activated stellate cells, endothelial cells and Kupffer cells which are associated with ALF (Example 1 of the Examples section which follows), the present invention in some embodiments thereof also contemplates targeting these cell populations for treating acute liver failure.

Thus, according to an aspect of the present invention, there is provided a method of treating acute liver disease in a subject in a subject in need thereof, the method comprising administering to the subject a therapeutically effective amount of a toxic agent attached to a targeting moiety for specifically targeting a cell selected from the group consisting of a stellate cell, an endothelial cell and a Kupffer cell, thereby treating the acute liver disease in the subject.

According to an additional or an alternative aspect of the present invention, there is provided a toxic agent attached to a targeting moiety for specifically targeting a cell selected from the group consisting of a stellate cell, an endothelial cell and a Kupffer cell for use in treating acute liver disease.

The term “targeting moiety”, as used herein, relates to a functional group which serves to target or direct the toxic agent or the composition comprising same described herein to a specific cell type (e.g. stellate cell, an endothelial cell and a Kupffer cell). Such targeting moieties include, but are not limited to antibodies, cell surface receptor, ligands, hormones, lipids, sugars and dextrans.

According to specific embodiments, the stellate cell, the endothelial cell and/or the Kupffer cell is a Myc-driven acute liver activated cell.

According to specific embodiments, the stellate cells have a Dcn+Rgs5+Lrat+Ecm1+ phenotype.

According to specific embodiments, the endothelial cells are sinusoidal endothelial cells having a Ptprb+Kdr+Clec4g+Vwf− phenotype.

According to specific embodiments, the Kupffer cells have a Ptprc+Timd4+Adgrel+Clec4f+Cd5l+ phenotype.

According to specific embodiments, the targeting moiety is an antibody.

The toxic agent may be covalently or non-covalently attached to the targeting moiety.

According to specific embodiments, the targeting moiety induces internalization of the toxic agent into the target cell.

Toxic agents are well known to the skilled in the art. Non-limiting Examples of toxic agent that can be used with specific embodiments of the invention include Pseudomonas exotoxin (GenBank Accession Nos. AAB25018 and S53109); PE38KDEL; Diphtheria toxin (GenBank Accession Nos. E00489 and E00489); Ricin A toxin (GenBank Accession Nos. 225988 and A23903)].

The agent of some embodiments of the invention (e.g. antibiotic, TLR-MYC pathway inhibitor, toxic agent) can be administered to the subject as a single treatment or in combination with other established or experimental therapeutic regimen to treat the acute liver disease (e.g., before, simultaneously or following) including, but not limited to supportive treatment, liver transplantation and other treatment regimens known in the art.

The agent of some embodiments of the invention (e.g. antibiotic, TLR-MYC pathway inhibitor, toxic agent) can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent (e.g. antibiotic, TLR-MYC pathway inhibitor, toxic agent) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., acute liver disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mouse models—8 weeks old C57BL6 male mice were injected intraperitoneally with 500 mg/kg body mass acetaminophen (APAP) in PBS or 300 mg/kg of thioacetamide (TAA) in PBS, 20 hours prior to sample collection. To avoid known circadian effects, all injections were performed between 1 pm and 2 pm. Control mice were injected with vehicle (PBS). For antibiotic treatment, mice were given a cocktail of ampicillin (1 g/l), kanamycin (1 g/l), vancomycin (0.5 g/l) and metronidazole (1 g/l) in drinking water for two weeks. MyD88 and Trif double knockout mice were 8 weeks old, males on C57BL6 background⁵⁵. All experimental procedures involving mice were approved by the local IACUC.

Human samples—All human studies were approved by the Weizmann Institute of Science Bioethics and Embryonic Stem Cell Research oversight committee, IRB approval number 699-1 and by the Rabin Medical Center Institutional Review Board, approval number: RMC-19-0816.

Liver cell isolation—Liver cells were isolated using a modified protocol by Mederacke and colleagues.¹⁴ Briefly, using a peristaltic pump a retrograde liver perfusion from inferior vena cava was performed with three solutions: first EGTA solution (8 g/l NaCl, 0.4 g/l KCl, 88 mg/l NaH₂PO₄.H₂O, 120 mg/l Na₂HPO₄.H₂O, 2.38 g/l HEPES, 0.35 g/l NaHCO₃, 0.19 g/l EGTA, 0.9 g/l glucose) for 2 minutes, second pronase solution (0.4 mg/ml protease in enzyme biffer solution (EBS): 8 g/l NaCl, 0.4 g/l KCl, 88 mg/l NaH₂PO₄.H₂O, 120 mg/l Na₂HPO₄.H₂O, 2.38 g/l HEPES, 0.35 g/l NaHCO₃, 0.42 g/l CaCl₂) for 5 minutes and third collagenase D solution (0.1 U/ml collagenase D in EBS buffer) for 7 minutes. Following, liver was dissected, placed in cold EBS solution, shaken vigorously with forceps for separation of single cells, followed by filtration through a 100 μm mesh. Hepatocytes were depleted by centrifugation at 30 g for 5 minutes. Cells were then collected by centrifugation at 580 g and resuspended in cold PBS. To enrich for stellate cells, cells with retinoid fluorescence in the Pacific Blue channel were sorted using a BD FACSAria III. Following, stellate cells were mixed with unsorted cells, spun down, resuspended in PBS with 0.04% BSA and counted using Neubauer chamber.

10× library preparation and sequencing—Single cells were captured and processed using the 10× Genomics Chromium 3′ Single Cell RNA seq protocol according to the manufacturers' manual. Subsequently, RNA was sequenced using NextSeq® 500/550 High Output Kit v2 (Illumina cat no. 20024906).

Measurement of monocyte infiltration—Livers harvested from mice were finely chopped with sterile scissors and digested with 4 ml of pre-warmed 0.4 mg/ml protease and 0.1 U/ml collagenase D (EBS buffer: 8 g/l NaCl, 0.4 g/l KCl, 88 mg/l NaH₂PO₄.H₂O, 120 mg/l Na₂HPO₄.H₂O, 2.38 g/l HEPES, 0.35 g/l NaHCO₃, 0.42 g/l CaCl₂) for 30 minutes at 37° C. while shaking Following, 10 ml of cold PBS was added and the suspension was filtered through a 100 μm mesh. To deplete hepatocytes, samples were centrifuged at 30 g for 5 minutes and the supernatant was transferred to new tubes. Cells were collected by centrifugation at 580 g. To lyse red blood cells, 1 ml of Gibco™ ACK Lysing Buffer was added and cells were incubated at room temperature for one minute. Subsequently, cold PBS was added and cells were collected by centrifugation at 580 g.

Receptors on the cells were first blocked with TruStain FcX anti-mouse CD16/32, then the cells were washed with FACS buffer (PBS without calcium and magnesium, 1% FCS), collected by centrifugation at 580 g and stained with CD45-PECy7 (Biolegend, 30F11, cat. 103114), F4/80-FITC (Serotec, Cl:A3-1, cat. G00018) and Ly6C-APC (Biolegend, HK14, cat. 128016) antibodies for 1 hour on ice. Finally, the cells were washed with FACS buffer, collected by centrifugation at 580 g, resuspended in FACS buffer, filtered through 40 μm mesh and measured on DB LSRFortessa. Data was analysed using FlowJo software.

Histology—Sample from the left lobe of the liver was fixed in 4% paraformaldehyde, embedded in paraffin, sectioned and stained with hematoxylin and eosin (H&E). Slides were scored by a blinded veterinary pathologist for necrosis and hemorrhage on a scale from 0 (healthy) to 5 (most severe).

MYC inhibition—MYC inhibitor KJ-Pyr-9 or vehicle control was injected intraperitoneally 2 hours following injection of APAP or PBS vehicle control. Briefly, 10 mg of KJ-Pyr-9 (Tocris, cat 5306) was dissolved in 1 ml of DMSO and then combined with Tween 80 and 5% dextrose 1:1:8 by volume. Mice were injected with 0.5 ml per 20 g, so the final dose of KJ-Pyr-9 was 25 mg per kilogram of body mass.

Inhibition of MAPK pathway proteins—The inhibitors listed in Table 1 hereinbelow were injected intraperitoneally 1 hour following injection of 500 mg/kg APAP or corresponding PBS vehicle. Briefly. The inhibitors were dissolved in 5% DMSO in PBS to a final injection volume of 400 μl for 20 g mouse. Control mice were injected with 5% DMSO in PBS vehicle.

TABLE 1
List of MAPK inhibitors

| inhibitor | target | Dose | reference |
|---|---|---|---|
| PF 06650833 | IRAK4 | 10 mg/kg | Wu et al. 2017 Blood |
| 5Z-7- | MAPK3K7 | 10 mg/kg | Zhou et al. 2018 J Cell |
| oxozeaenol | (Tak1) | | Mol Med |
| TC-S 7006 | MAPK3K8 | 10 mg/kg | Vyrla et al. 2016 J Immunol |
| | (Tpl2) | | |
| PD0325901 | MEK1/2 | 5 mg/kg | Wu et al. 2011 J Clin Invest |
| | (Erk1/2) | | |
| SB203580 | p38 | 25 mg/kg | Mannangatti et al. 2015 |
| | | | J Biol Chem |
| Nec1 | RIP1 | 2 mg/kg | Berger et al. 2015 Cell |
| | | | Death Discov |

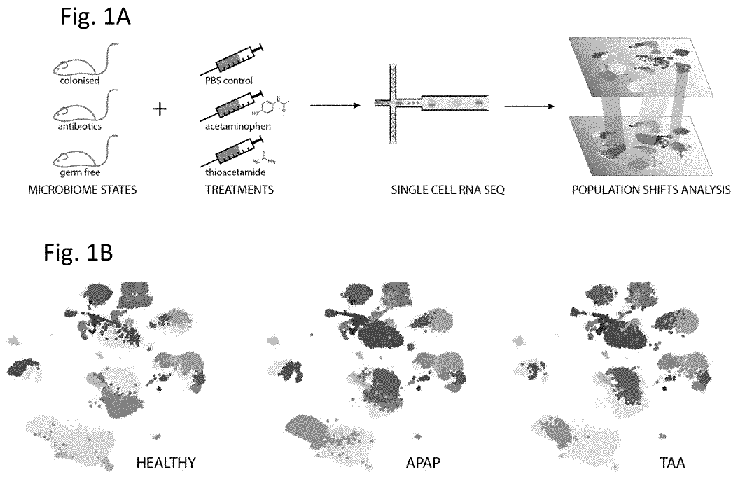

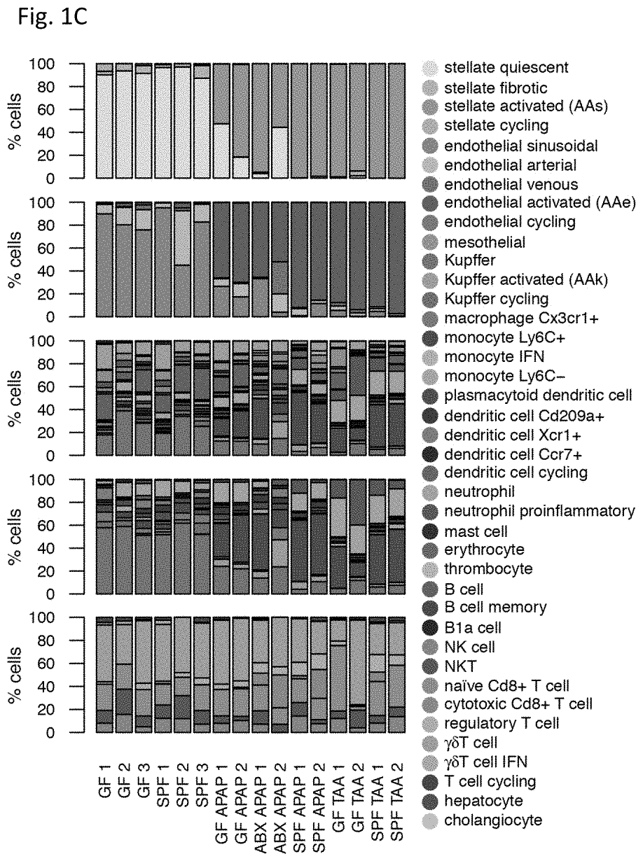

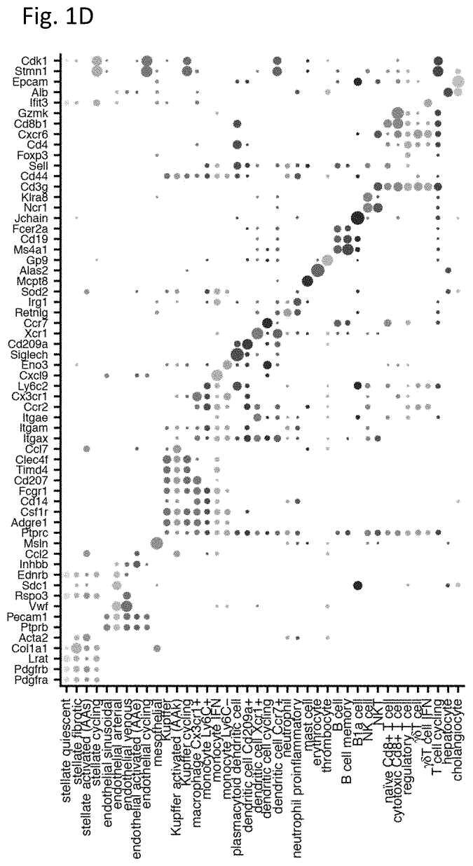

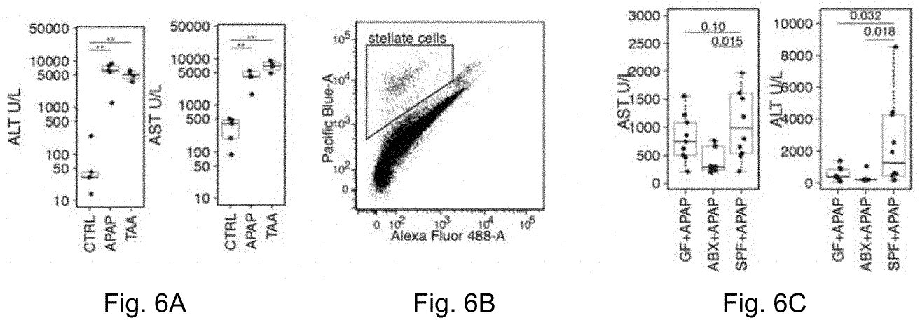

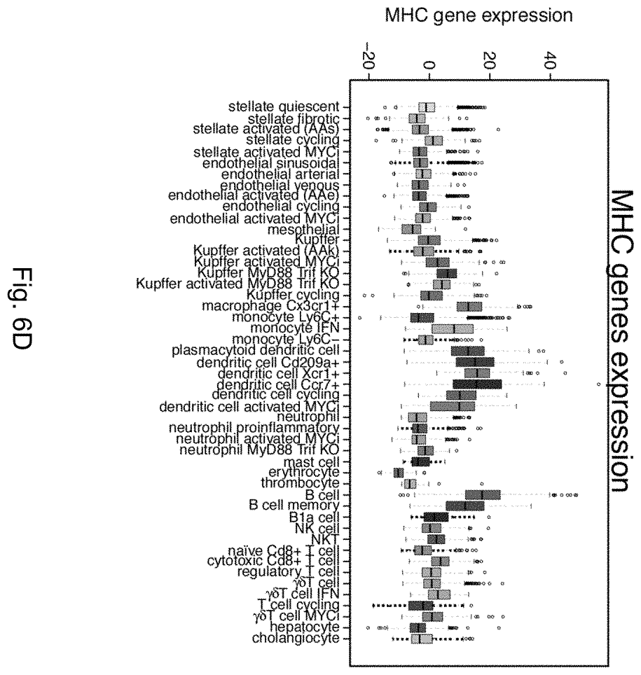

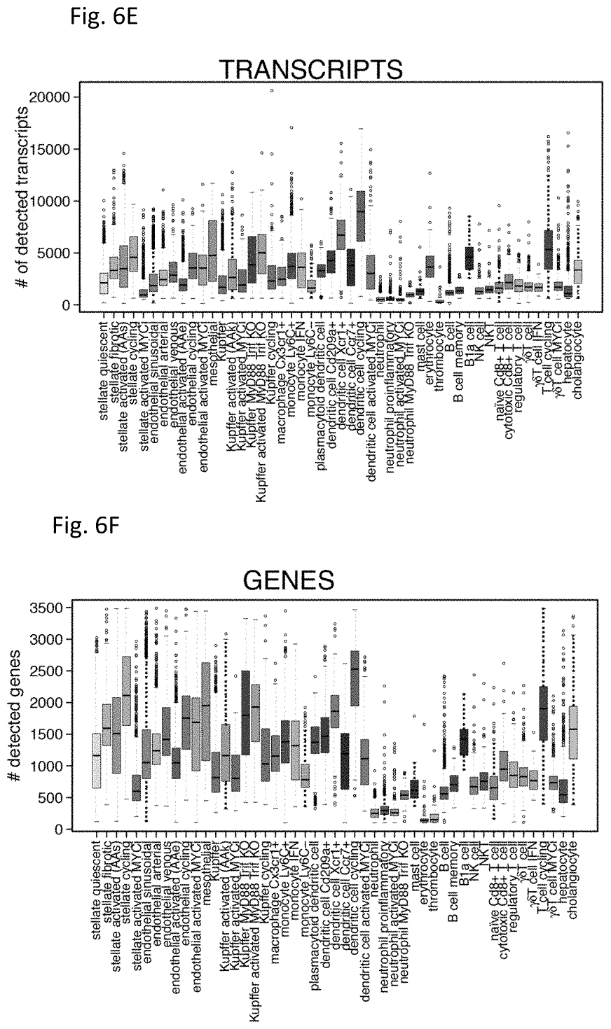

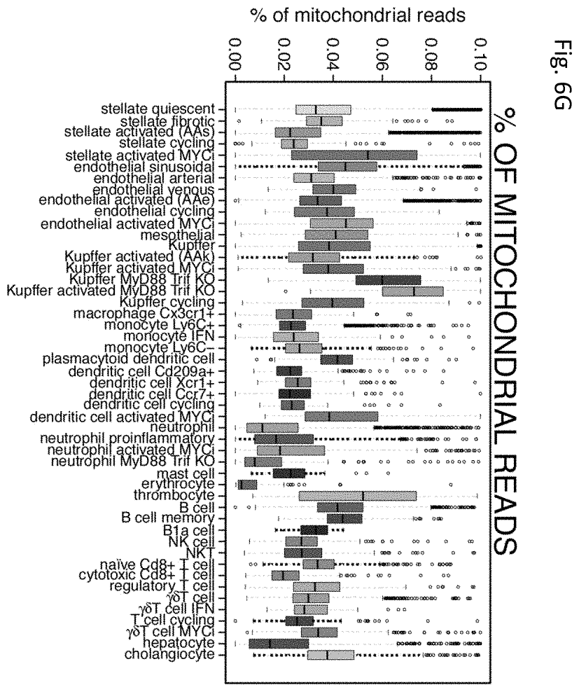

Liver enzymes activity measurement—Blood serum ALT and AST activity was determined using a Liver-1 test on Arkray SPOTCHEM EZ SP-4430 for first samples (shown in FIG. 6A) to validate the model (FIG. 1A). All following measurements were effected using Roche Cobas 111 Serum analyser.

16S targeted bacterial composition profiling—Colon and small intestine contents were collected post mortem, flash frozen in liquid nitrogen and stored at −80° C. DNA was extracted from the samples with Invitrogen PureLink Microbiome DNA Purification Kit according to the manufacturer's protocol. The V4 fragment of the 16S gene was amplified using AATGATACGGCGACCACCGAGATCTACACGCTTATGGTAATTGTGTGCCAGCMGCC GCGGTAA (SEQ ID NO: 1) and CAAGCAGAAGACGGCATACGAGATAGTCAGCCAGCCXXXXXXXXGGACTACNVGG GTWTCTAAT (SEQ ID NO: 2) primers, where XXXXXXXX denotes a barcode. PCR was performed using TaKaRa Ex Taq® DNA Polymerase with 120 ng of genomic DNA input, and 0.4 μM of each primer in 25 μl reactions. PCR amplification, and lack of it in negative controls, were verified with agarose gel electrophoresis. Samples were purified using 0.8×SPRI beads (Agencourt AMPure XP), pooled equimolarly and sequenced with Miseq V2 kit (2×250 cycles).

Quantification of microbial products in portal serum—The following PRR reporter cell lines were obtained from Invivogen (HEK-Blue TLR and NLR reporter cell lines): TLR2, TLR3, TLR4, TLR5, TLR7, TLR9, NOD1, NOD2. Portal vein plasma samples that were aseptically collected from mice, were added to reporter cell lines and incubated with HEK-Blue detection medium (Invivogen) according to the manufacturer's instructions.

Immunohistochemistry—The 4 μm thick sections were deparaffinized, rehydrated, treated for 30 minutes with 6 ml H₂O₂+200 ml 70% methanol+2 ml HCl in order to block endogenous peroxidase activity, and washed in PBS. Antigen retrieval was effected using citric acid. The sections were incubated with blocking solution, followed by AB blocking kit and incubated over night with anti-cMYC monoclonal antibody (13-2500, 1:25, Invitrogen). The sections were then incubated with mouse biotin, followed by the ABC kit, and stained with DAB and haematoxylin. Following, the sections were dehydrated, cleared in xylene and cover slipped. The sections were viewed using a microscope under ×20 magnification to monitor the color of nucleus. A positive cell was considered to have a red-brown colored nuclei. The number of total positive and negative nuclei was determined automatically by ‘Image pro’ computer program, followed by training the software on couple manually selected positive and negative cells.

Western Blotting—Liver tissue was excised and homogenized in RIPA buffer containing protease and phosphatase inhibitors, incubated for 20 minutes at 4° C. and centrifuged for 5 minutes, 2700 g at 4° C. The supernatant was further centrifuged for 30 minutes, 15000 g at 4° C. Samples were run on 12% acrylamide gels and transferred onto nitrocellulose membranes. Western blot analysis was performed using anti-cMYC monoclonal antibody (13-2500, 1:1000, Invitrogen), anti-cMyc-Phospho-Ser62 (PAS-104729, 1:1000, Invitrogen), Goat anti-mouse HRP (115-035-205, 1:5000, Jackson Labs) and Goat anti-rabbit HRP (111-035-003, 1:5000, Jackson labs). Western blot imaging and band intensity quantification were performed using Gel Doc XR+ system (Bio-rad).

Single Cell RNA Sequencing Data Analysis—

Mapping: Single cell RNA sequencing data were demultiplexed, mapped to the GRCm38 mouse genome and UMIs were counted using the Cell Ranger Single-Cell Software Suite 2.1.1.
Filtering and doublet removal: Cells with less than 100 detected transcripts and more than 10% mitochondrial reads were removed. Clustering analysis was used to identify populations of thrombocytes, erythrocytes, neutrophils and mast cells. Following, a second filtering was effected using 600 detected transcripts. This filtering step did not include the above mentioned cell populations, as these cells have small transcriptomes and thus would be lost. The second step was necessary, as there were many low quality cells with low threshold. Following, doublets were identified by finding clusters of cells expressing gene expression patterns of two cell types at the same time. Marker sets used were Dcn, Colec11, Ecm1, Cxcl12, Sod3, Angptl6, Rgs5, Reln, Tmem56, Rbp1, G0s2, Rarres2, Acta2, Tagln for stellate cells; Gpihbp1, Aqp1, Clec4g, Dnase113, Fabp4, Ptprb, Kdr, Gpr182 for endothelial cells; Alb, Ttr, Ambp, Rbp4, Cyp2e1, Spp1 for hepatocytes; Cd3e, Cd3g, Cd3d, Lat, Thy1, Cxcr6, Nkg7, Cd4, Cd8b1, Klra4, Ncr1, Gzmb, Lck, Txk, Ms4a4b, Ccl5 for T cells; Cd79b, Cd79a, Ms4a1, Siglecg, Fcmr, Cd19, Fcer2a for B cells; Adgre1, Cd51, Clec4f, Timd4, Folr2, C1qa, C1qb, C1qc, Vsig4, Xcr1, Cd209a, Itgax, Siglech, Chil3, F13a1, S100a4, Lgals3, Gda, S100a9, Retnlg for macrophages, DCs and neutrophils; Alas2, Hbb-bt, Hbb-bs, Hba-a2, Hba-a1 for erythrocytes.
Clustering—All cells from all 22 samples were first clustered using R package Seurat v2.3.4 FindClusters function⁵⁶. Genes that were present in less than 3 cells were removed and then highly variable genes were identified as having mean of non-zero values between 0.0125 and 3 and standard deviation higher than 0.5. Dimensionality reduction was effected with PCA and first 50 PCs were used for clustering.

As the liver population is very complex, it was decided to perform stepwise clustering. First, expression of Ecm1, Ptprc, Ptprb, Epcam, Msln and Alb in the obtained clusters was determined. Based on this expression the cells were divided into 7 groups: stellate cells, immune cells, endothelial cells, mesothelial cells, cholangiocytes, hepatocytes and cycling cells. The immune cells were reiterated in the same way once again and based on expression of Agdre1, Cd5l, Ncr1, Cd3e, Cd79b, Retnlg, Cx3cr1, and Stmn1 were split into 4 groups: B cells, T cells and NK cells, neutrophils and remaining immune cell types. Within these groups, cells were clustered using Seurat FindClusters algorithm.

Cell type markers identification and annotation—For each cluster, marker genes were identified with FindMarkers function and based on cross-checking identified markers with known marker genes and comparison to the ImmGen database clusters with cell types were annotated. Many of the clusters were assigned to macrophages or dendritic cells, hence a key marker gene was added to their cell type description to make it easier to the wider audience to compare to other data. Clusters that represented the same cell type were merged: i.e. Ly6C monocytes and Cxcr6 T cells.
Functional analysis of cell populations—Gene ontology analysis and TF binding motif analysis was performed using g:Profiler⁵⁷ with default settings, multiple hypothesis testing adjustment using all mouse genes as a background control. Log 10 of FDR adjusted p-values were plotted as bar plots. For heatmaps gene ontology lists were obtained from Ensembl BioMart.
Ligand-Receptor interactions mapping—Differentially expressed genes between quiescent stellate, sinusoidal endothelial and Kupffer cells and their corresponding activated counterparts were filtered for ligands from database accompanying paper by Ramilowski et al.³⁴. Following, corresponding receptors were identified in the database and both normalized and scaled expression of ligands and receptors were plotted as balloon plots.
Comparison of gene expression between clusters—To compare gene expression between clusters, such as quiescent stellate cells vs. acute stellate cells, Seurat FindMarkers function was used defining identity of both clusters.
Comparison of gene expression within a cluster between conditions—To compare gene expression between samples, such as activated stellate cells in GF APAP-induced versus activated stellate cells in SPF APAP-induced mice, pseudobulk was calculated by adding reads from all cells within each cluster in a sample. Following, DESeq2 using default parameters was used to find differential expression.⁵⁸
Diffusion maps—Diffusion maps were calculated using destiny R package⁴². First, Ly6C-positive monocytes were clustered revealing 4 different subclusters. Using Seurat FindMarkers function 50 top specific genes were identified for each cluster. Normalized data was filtered for genes specific for these subsets and this data was used to calculate diffusion maps, using euclidean distances, local scale parameter sigma, without rotated eigenvalues and taking 10 nearest neighbors.

16S V4 amplicon sequence analysis—16S amplicon sequences were analysed using Qiime2⁵⁹. Sequencing reads were demultiplexed with demux plug-in. 31 poor quality bases were trimmed from the reverse read, and one base from forward read, combined, denoised and amplicon sequence variants (ASVs) were called with dada2. Sequences were aligned using Mafft, masked and a phylogenetic tree was constructed using phylogeny fasttree. Following, reads were rarefied to 20000 reads per sample. Taxonomic assignment to ASVs was effected using feature-classifier classify-sklearn and Greengenes 13_8 99% OTUs. Differential abundance analysis was effected with Wilcoxon rank sum test and Benjamini-Hochberg FDR correction.

Data availability—The scRNAseq sequencing data has been deposited at the ArrayExpress accession number E-MTAB-8263 and 16S sequencing data to ENA accession number ERP116956.

### Example 1

### Cellular and Molecular Changes in Acute Liver Failure

ALF was induced in adult 8-week-old C57BL6 mice with acetaminophen (APAP) or thioacetamide (TAA) (FIG. 1A). Both drugs induced severe and fulminant liver damage, manifesting as acute elevation in alanine and aspartate aminotransferase activity in blood serum (FIG. 6A). Of note, both TAA and APAP elicit oxidative stress through similar mechanisms^{2,11}, and an ensuing intense liver inflammation further contributing to liver damage¹². To examine potential microbiome contribution to functional responses of cellular subsets, ALF was also induced by APAP or TAA following depletion of the microbiome using a two-week wide-spectrum antibiotic treatment (namely Ampicillin, Neomycin, Metronidazole and Vancomycin in the drinking water)¹³. To control for possible direct antibiotic impacts on liver physiology and ALF, ALF was also induced in germ-free mice, which are devoid of a microbiome (FIG. 1A).

To profile the hepatic non-parenchymal cellular populations in naïve, microbiome-depleted, and ALF settings, liver cells were isolated and hepatocytes were depleted from the sample by centrifugation¹⁴. Half the resultant cellular fraction was further enriched for HSCs by flow cytometry gating on intrinsic retinoid fluorescence of this cell population (FIG. 6B)¹⁴. The remaining half of the hepatic cellular fraction remained unaffected to enable unbiased quantification using single cell genomics. Using droplet-based single cell RNA-seq, 6399 cells from 8-week-old male healthy C57BL6 mice housed in specific pathogen free (SPF) conditions; 6160 cells from mice housed in germ free (GF) conditions; 11154 cells from APAP-treated mice housed in GF, SPF and treated with oral antibiotics (ABX); and 7145 from TAA-treated mice housed in GF and SPF conditions were analyzed (FIG. 1B). Using hierarchical clustering, 40 different cell populations were identified, that could be divided into 6 major types immune cells, endothelial cells, HSC, hepatocytes, cholangiocytes and mesothelial cells, collectively resulting in a high-resolution liver cell atlas (data not shown). Importantly, while microbiome depletion in GF mice results in attenuated ALF (FIG. 6C), no significant differences were found in the gross abundance of resident hepatic cell populations between GF and SPF mice both in health and disease models (FIG. 1C). Cell clusters were annotated using conventional markers and by comparison of their specific gene expression patterns to the Immgen database¹⁵ (FIG. 1D).

Within stellate cells, four distinct populations were identified. Based on their markers, they were classified as Lrat^{high} quiescent stellate cells, Col1a1-positive fibrotic stellate cells, Acta2-positive ALF activated stellate cells (referred to as AAs) and cycling stellate cells. In the endothelial cell population, three clusters bearing different transcriptional signatures depending on their localization were identified. The most abundant liver sinusoidal endothelial cell (LSEC) population was positive for Dnase113, Clec4g and Fcgr2b. The two additional smaller populations both expressed von Willebrand factor gene (Vwf), with one co-expressing Sdc1, Pecam1 and Cd34, suggesting that it originates from the central vein; and the other co-expressing Rspo3, Fmo2 and Gpm6s, suggesting that it is located in arteries¹⁶. Furthermore, a cycling endothelial cell subpopulation, as well as population of ALF-activated endothelial (referred to as AAe) cells were identified and are described in details hereinbelow. Within immune populations the highest heterogeneity was found among dendritic cells, macrophages and T cells. Four dendritic cell clusters were identified based on high expression of MHCII (FIG. 6D) and lack of expression of Adgre1 (coding for F4/80), including (in descending order of abundance) plasmacytoid dendritic cells (pDC), Cd209a-positive DCs (DC2), Xcr1-positive DCs (DC1) and migratory Ccr7-positive DCs^{17,18}. Interestingly, DC populations with similar markers were recently shown in the lungs¹⁹. Two populations of Kupffer cells were identified, one quiescent and one ALF-activated Kupffer (referred to as AAk) cells, that could be distinguished from other Adgre1-positive macrophages (coding F4/80) by their expression of Timd4 and Clec4f²⁰. Four additional populations of macrophages were also identified: two populations of Ly6C-positive monocytes, one expressing interferon response genes (described in details hereinbelow), liver-repair associated macrophages (Cx3cr1-positive, Ccr2-negative) and a Ly6C-low population of monocytes that specifically expresses Eno3 and Ace genes coding for Enolase 3 and Angiotensin-converting enzyme, respectively. Ace-expressing macrophages were described in other contexts²¹. As previously shown in the human liver, both αβT and γδT cells were abundant in the mouse liver²². Among the αβT population, regulatory T cells expressing Cd4, Foxp3 and Ikzf2 and populations of naïve and cytotoxic Cd8-positive cells were identified.

Response to ALF-inducing toxic insult was characterized by initial changes in resident liver cells and a subsequent recruitment of infiltrating immune cells²¹. Indeed, in both models new activated cellular states arising within the stellate, endothelial and Kupffer cell populations were observed (FIGS. 1C-D). This increase in the activated state, coincided with a similar decrease in abundance of the respective non-activated cell states, suggesting that these cells are activated counterparts rather than recruited populations. An increase was also observed within Ly6C-positive monocytes in both TAA and APAP models and in neutrophil populations in response to TAA.

In the next step, gene expression profiles of activated resident cell types upon induction of ALF as compared to their steady state configuration were determined.

ALF-Activated Stellate (AAs) Cells

Previously, HSC states were characterized mainly in health and chronic disease models such as NAFLD and fibrosis, as reported both in bulk and to a limited extent also on the single cell level^{23,24}. Importantly, Col1a1 and Acta2 are considered hallmark markers of HSC in disease, often called ‘myofibroblasts’²²⁻²⁴, suggestive of two features of stellate cells in disease: a contractile (‘myo’) phenotype mediated by expression of stress fiber genes, and an extracellular matrix-secreting (‘fibroblast’) phenotype, especially of collagens²⁵.

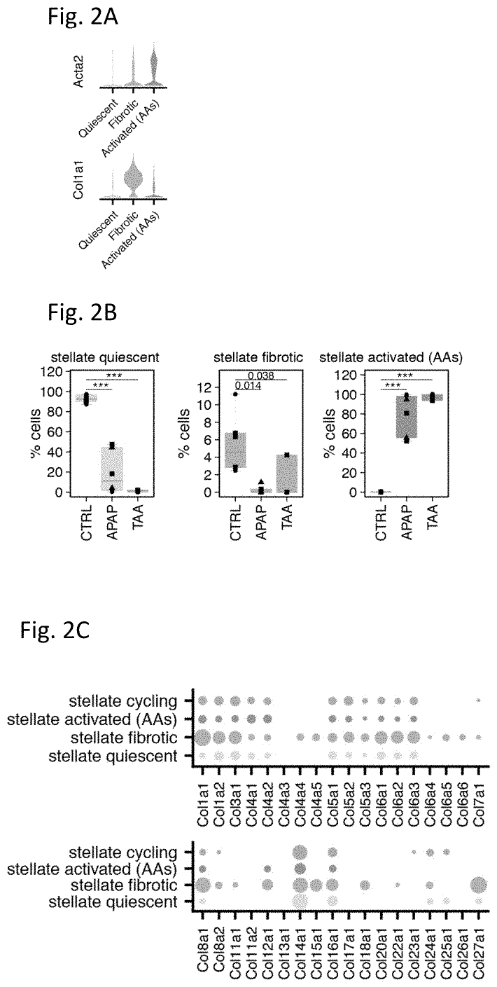

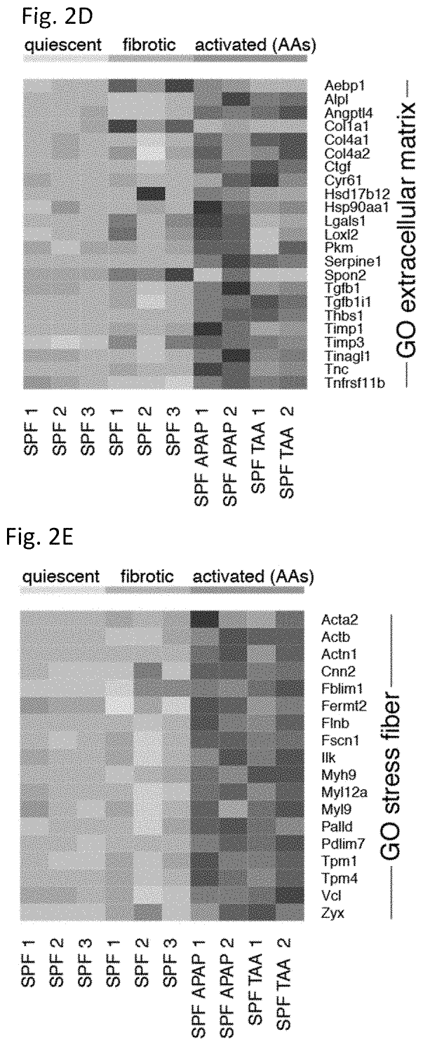

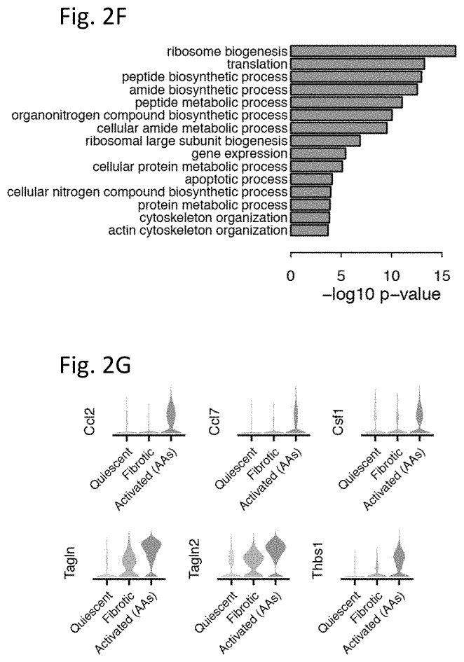

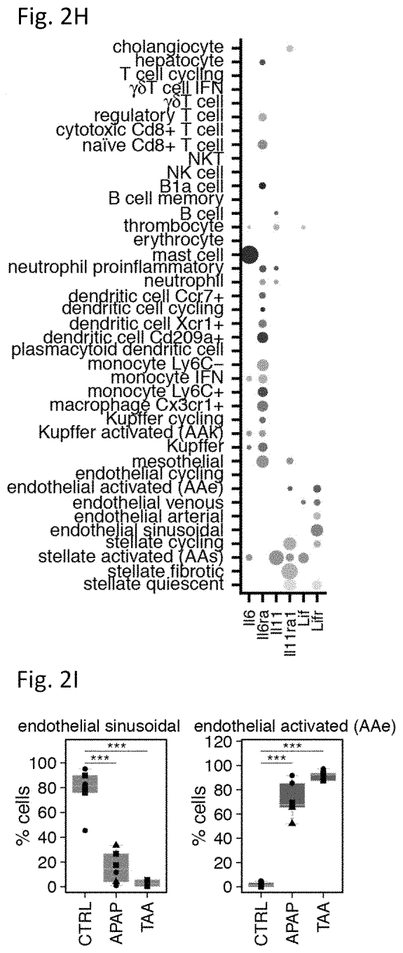

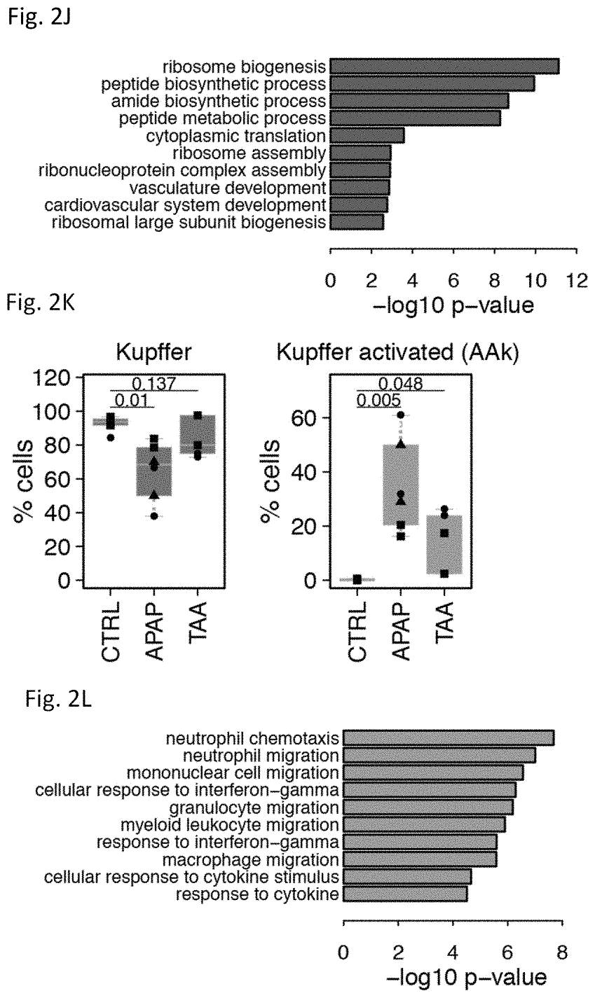

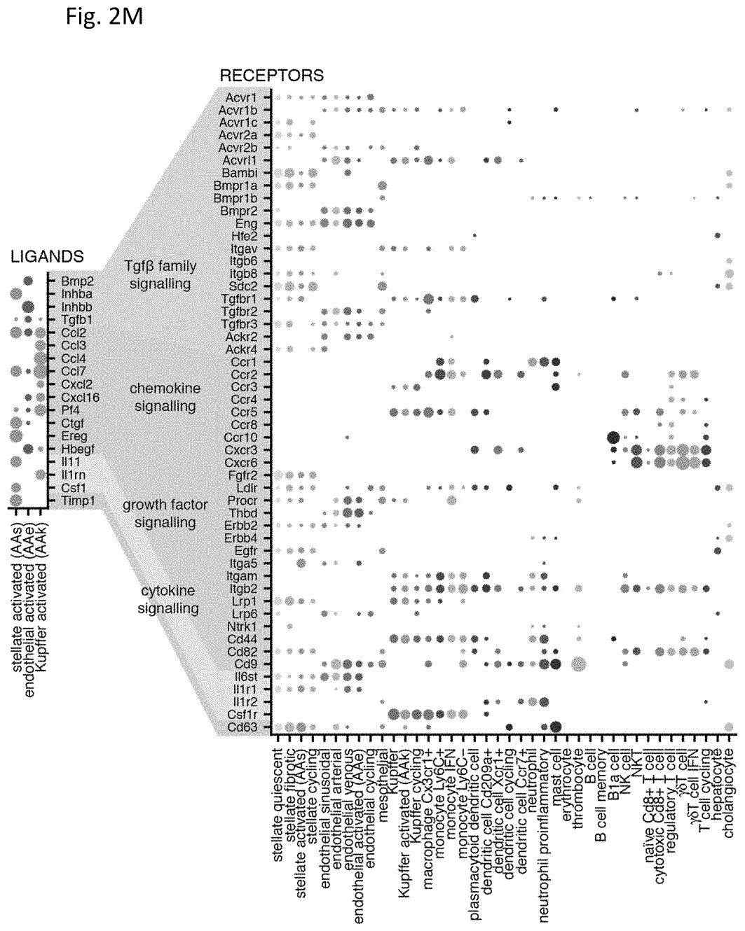

Interestingly, in both ALF models, expression of Col1a1 and Acta2 was mutually exclusive and only Acta2-positive AAs cells, but not Col1a1-positive fibrotic cells, were strongly upregulated upon ALF-induced activation (FIG. 2A). In both the APAP and the TAA models, on average 87.3% of the stellate cells assumed an Acta2-positive AAs state; while the population of Col1a1-positive fibrotic stellate cells, that comprises on average 5.4% of cells in healthy mice, similarly to the population of quiescent HSCs, was significantly downregulated (FIG. 2B). During ALF, the fibrotic stellate cell population assumed a higher expression of collagens in comparison to quiescent HSCs as well as in comparison to AAs (FIG. 2C). Interestingly, collagen IV genes, Col4a1 and Col4a1, were an exception and expressed by the ALF-activated stellate cells, rather than by fibrotic population. Similarly, several genes from gene ontology term extracellular matrix (GO:0031012) were also upregulated in AAs cells in comparison to quiescent cells. (FIG. 2D). Furthermore, several genes belonging to the GO term ‘stress fiber’ were upregulated in ALF-activated stellate cells, including the hallmark gene Acta2 (FIG. 2E). Collectively, these results suggest that, in addition to a quiescent stellate cell state, there are at least two distinct phenotypes that stellate cells can assume, namely a Col1a1-expressing fibroblast state and an immunomodulatory, contractile Acta2-positive state. At least in the context of the tested ALF models, these transcriptional programs do not coexist within one cell.

To further dissect function of AAs cells differential expression analysis between quiescent and activated stellate cell populations was performed which identified 642 genes induced by the toxic ALF insult. Gene ontology analysis of genes upregulated in AAs revealed multiple terms related to gene expression and translation (FIG. 2F), suggestive of a potentially increased need for protein production in these cells. This observation coincided with the higher number of detected genes and transcripts in the activated population (FIGS. 6D-E), including chemokines, such as Ccl2, Ccl7, cytokine Csf1, extracellular matrix components and modifiers including transgelins (Tagln, Tagln2) and thrombospondin 1 (Thbs1) (FIG. 2G). One of the highly expressed families of cytokines expressed by AAs during ALF were members of interleukin-6 family, including Il6, Il11 and Lif^{26,27}. Interestingly, receptors for these interleukins were expressed by different cell types, suggesting that IL-6 signaling may occur in immune cells, mesothelial cells and hepatocytes, IL-11 signaling may autocrinally occur in stellate cells and in cholangiocytes, and LIF signaling may occur in stellate cells and endothelial cells. Collectively, this suggested ALF-associated IL6 family program may represent a possible ‘division of labor’ in cellular signaling (FIG. 2H).

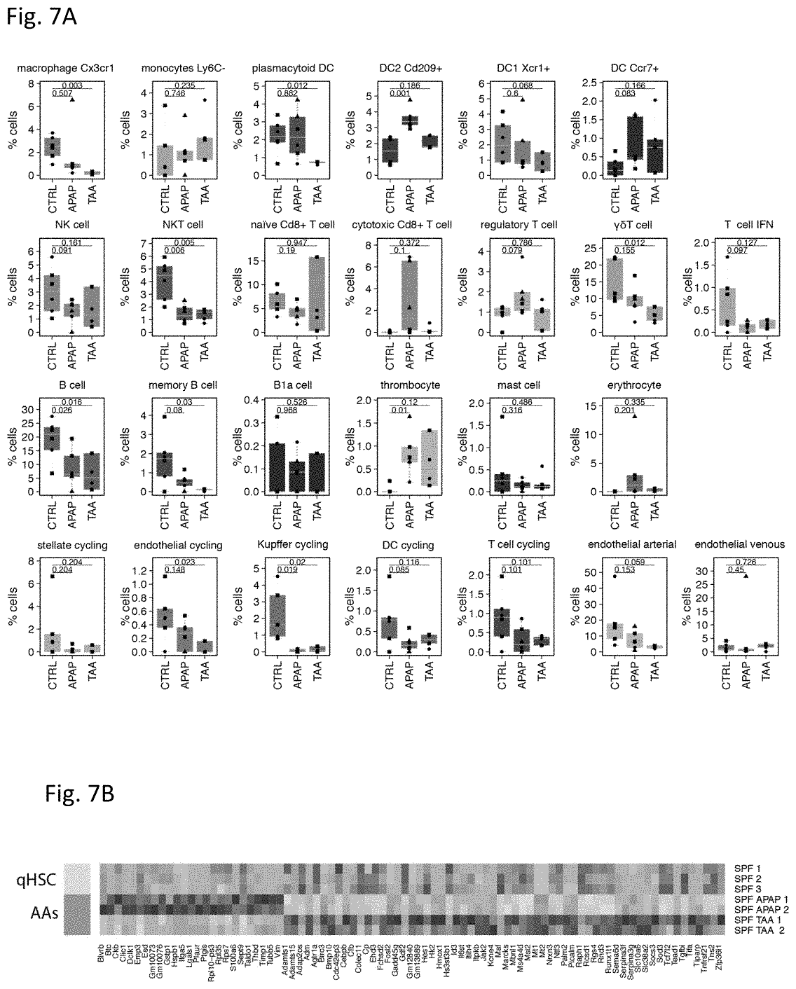

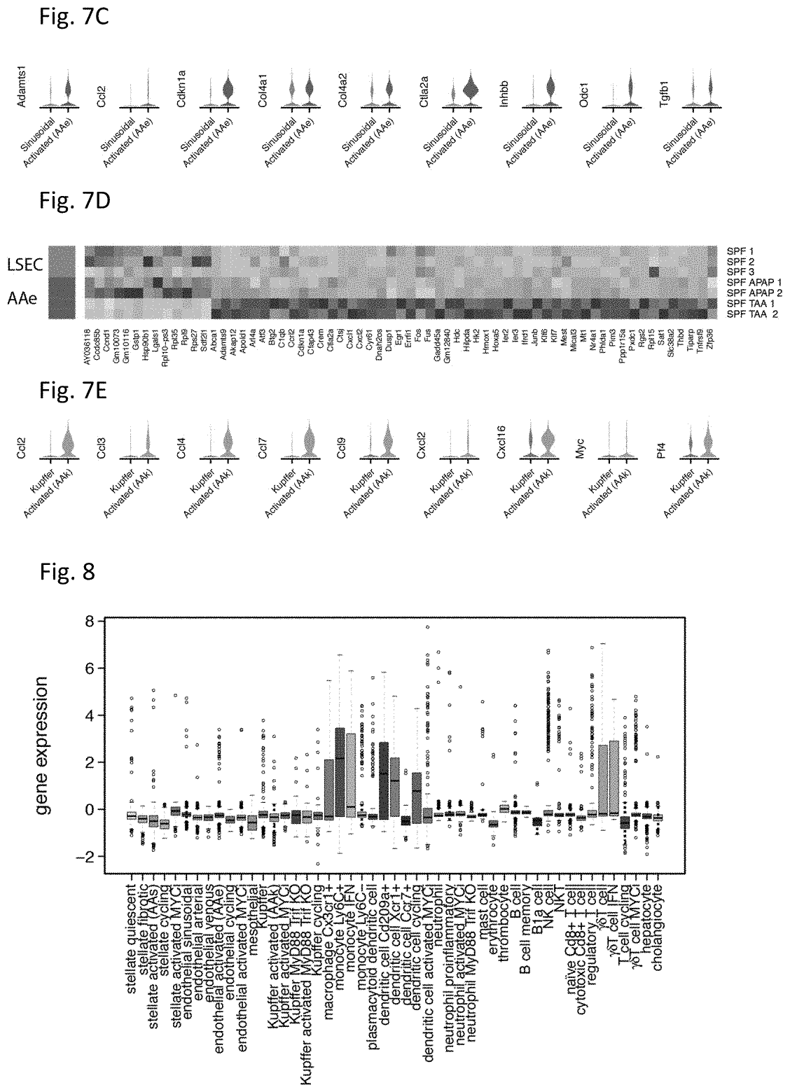

GO enrichment analysis of AAs also revealed terms associated with cell death. Trp53 and Cdkn1a, encoding p53 and p21, respectively, were found among the upregulated genes within these terms. In the presence of stress, p53 induces expression of Cdkn1a gene that triggers cell cycle arrest leading to senescence or apoptosis²⁸. Increased cellular transcriptional activity, coupled with markedly induced cytokine secretion collectively suggested that ALF-associated AAs cells may feature senescence rather than apoptosis²⁹. Moreover, several of the AAs upregulated genes belong to what was previously described as a senescence-associated secretory phenotype (chemokines, Timp1, Ereg)²⁹. Interestingly, a trend towards lower number of proliferating cells was noted in ALF in all resident populations (stellate, endothelial, Kupffer, dendritic and T cells), further supporting the notion that liver cells undergo cell cycle arrest (FIG. 7A).

Importantly, AAs cells in ALF induced by either APAP or TAA clustered together, suggesting that the differences in stellate cell activation states between these conditions are rather minor. To examine potential molecular differences between the two models, differential expression with DESeq2 using pseudo-bulk samples from single cell populations in the SPF APAP and SPF TAA samples was effected which identified 85 significantly differentially expressed genes (FIG. 7B). 24 of these genes which overlapped with the stellate cell activation signature, e.g. Itga5 and Timp1 were higher in APAP, while genes related to stress response, such as Mt1 or Mt2 were higher in TAA. Together, this suggests that the key transcriptional changes involving cytokines and extracellular matrix proteins are similarly upregulated in ALF regardless of the liver insult.

ALF-Activated Endothelial (AAe) Cells

The functions of liver endothelial sinusoidal cells span beyond building blood vessels and forming a barrier between blood circulation and the liver. Together with αSMA-positive (encoded by Acta2) contractile AAs cells, endothelial cells regulate blood flow in the liver through vasoconstriction, form a barrier for molecules and immune cell liver trafficking through regulation of fenestration and partake in blood clearance through endocytosis³⁰. Similar to stellate cells, on average 79.9% of liver sinusoidal endothelial cells assumed an activated phenotype upon ALF induction (FIG. 2I). Interestingly, venous and arterial endothelial cells did not exhibit a similarly strong transcriptional activation (FIG. 7A). AAe cells assumed immunomodulatory functions, by expressing the chemokine Ccl2, the Tgfβ family members Tgfb1 and Inhbb, as well as extracellular matrix remodeling genes such as Adamts1, Tgm2, Col4a1 and Col4a2 (FIG. 7C). Gene ontology term enrichment analysis of 254 upregulated genes in AAe revealed terms related to gene expression and terms associated with vascular remodeling (FIG. 2J). Lower levels of angiopoietin receptor Tek and Wnt2 were seen in AAe, a phenomenon previously associated with liver injury and hepatic regeneration^{31,32}. Collectively, this suggested that endothelial cells are active participants in both the ALF-associated immune response, extracellular matrix remodeling and regenerative processes in this model. Comparison of AAe cells between the APAP and TAA models revealed 65 differentially expressed genes, including several members of the AP-1 family transcription factors, such as Fos and Jun, that function in stress response; and cytokines including Cxcl1 and Cxcl2. This suggests that in the tested settings TAA may elicit more oxidative stress than APAP³³ (FIG. 7D).

ALF-Activated Kupffer (AAk) Cells

Due to the high complexity of macrophage populations in the liver, and accompanying challenges to dissect functions of subsets due to impurities of the subpopulations, the role of Kupffer cells in ALF remains unclear to date. ALF was associated with activation of on average 27.8% of Kupffer cells (FIG. 2K). Gene ontology analysis of 283 upregulated genes revealed terms related to chemotaxis, cell migration, immune response and apoptosis (FIG. 2L). The chemotaxis and cell migration terms represent AAk cells' expression of a battery of chemokines: Ccl2, Ccl3, Ccl4, Ccl6, Ccl7, Ccl9, Ccl12, Cxcl2, Cxcl16 and Pf4 (encoding CXCL4) (FIG. 7E). Similar to what was observed in stellate cells, apoptosis-related terms were associated with cell cycle arrest, as hallmarks of apoptosis such as decrease in number of transcripts or changes in percentage of mitochondrially encoded transcripts were not observed (FIGS. 6E-G). The activation of Kupffer cells was similar between APAP and TAA with Hmox1 identified as the only differentially expressed genes between the two disease models.

Communication networks between cells maintain a balance between co-residing cells during homeostasis and are frequently altered during disease development. To explore some liver cell-to-cell communication motifs in steady-state and ALF, first differentially expressed genes between AAs, AAe and AAk and their quiescent equivalents were filtered for ligands from dataset curated by Ramilowski et al.³⁴. 18 ligands that can be grouped into categories of Tgfβ ligands, chemokines, cytokines and growth factors were identified. Following, matching receptors for these ligands were identified. Due to redundancy in the ligand-receptor interactions (for example Ccl2 chemokine was shown to bind to Ccr1, Ccr2, Ccr3 and Ccr4) the receptors were grouped into the same functional categories as ligands, yielding major signaling modules: chemokines target mostly immune compartment, Tgfβ target mainly stellate and endothelial cells, while growth factors and cytokines seem to potentially affect all the cell types (FIG. 2M).

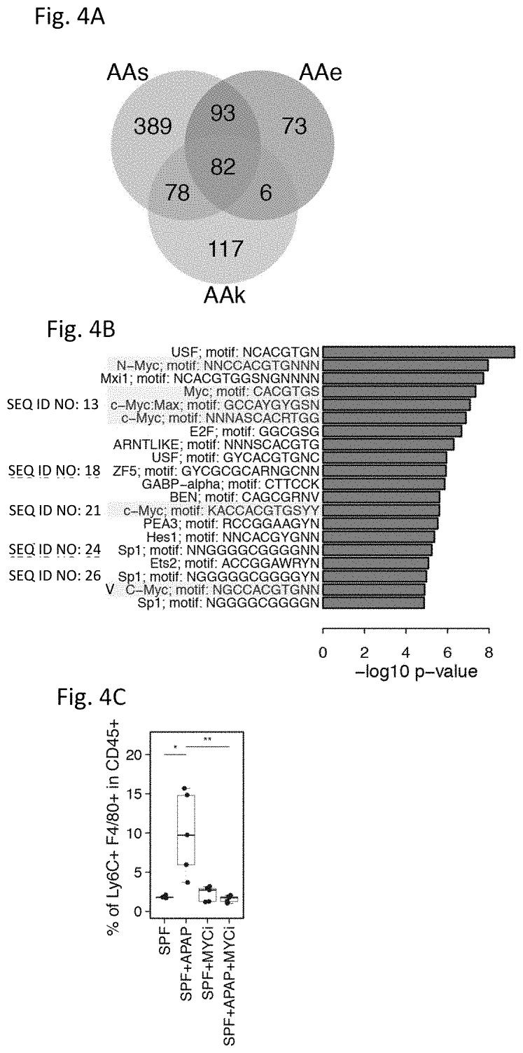

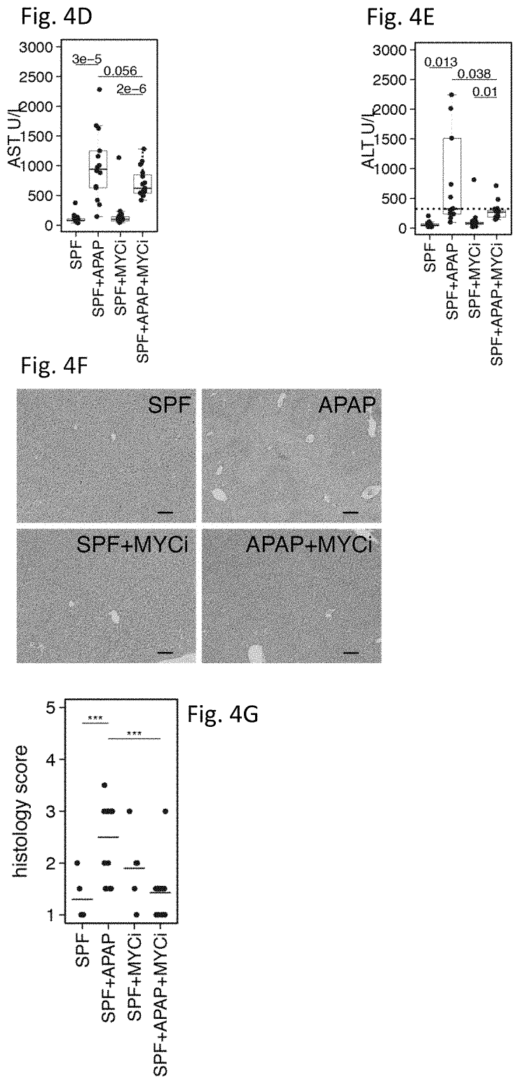

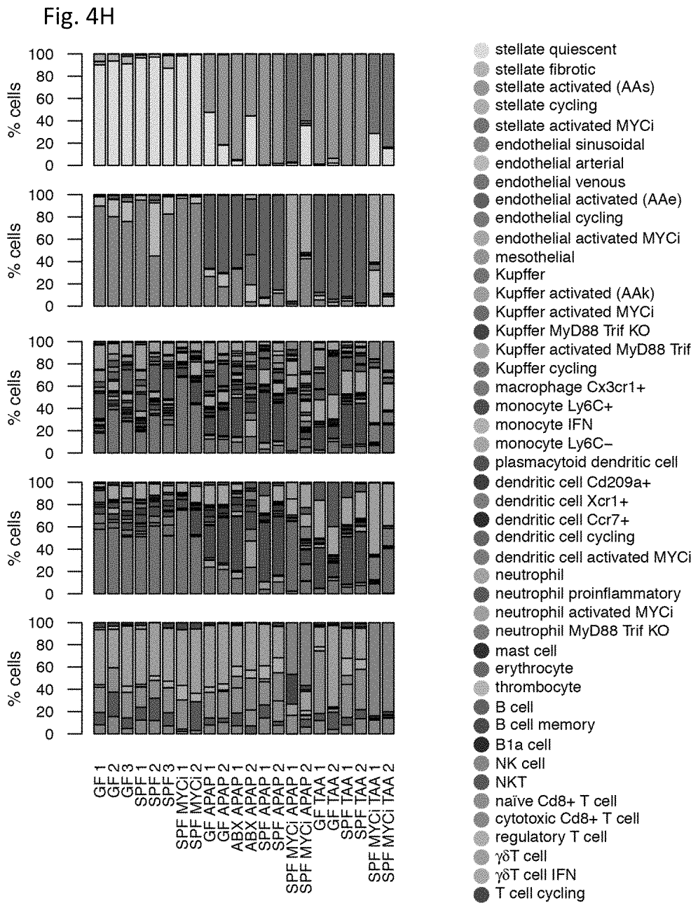

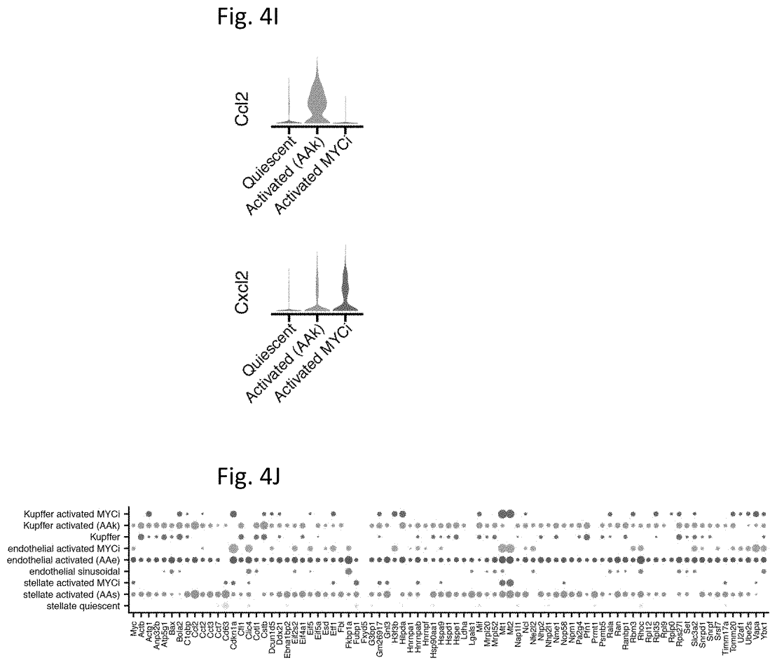

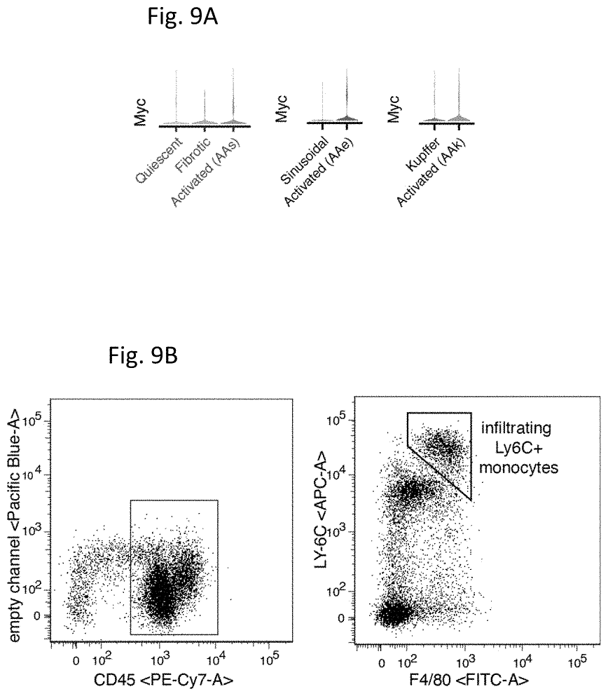

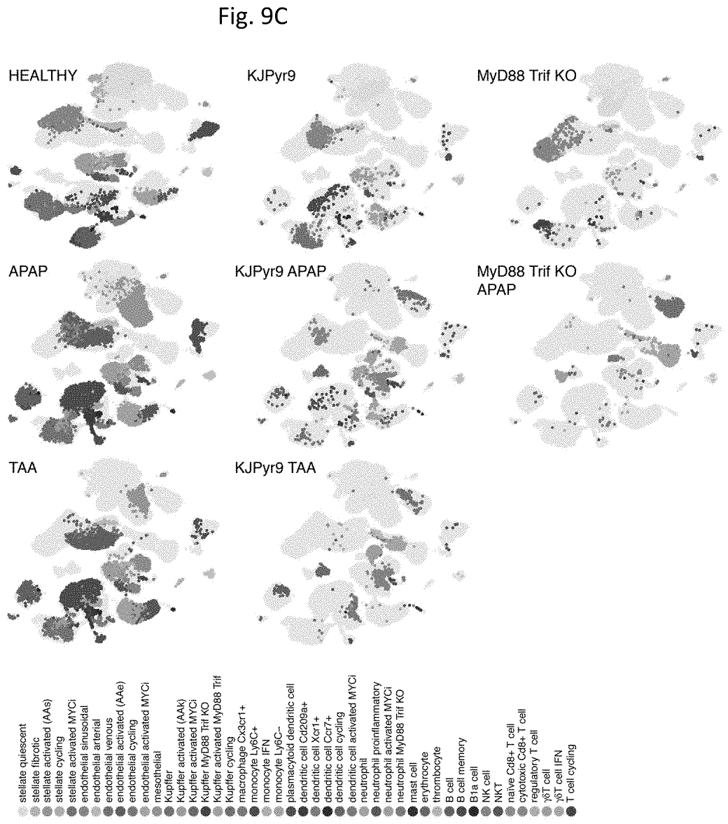

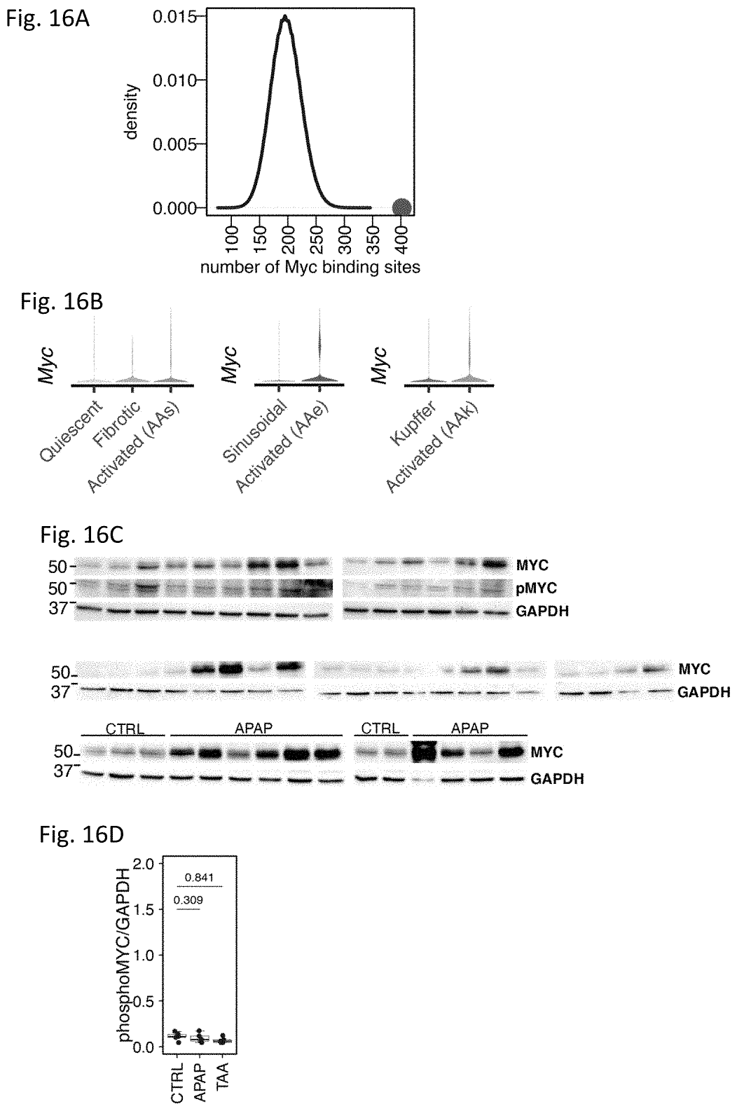

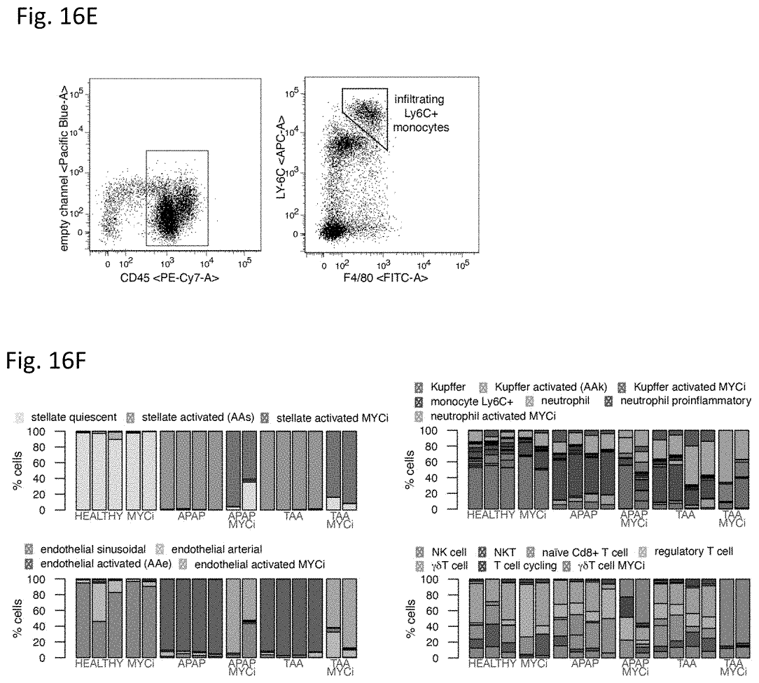

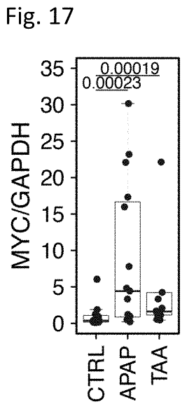

Careful examination of activation signatures of stellate, endothelial and Kupffer cells led to an observation that some of the upregulated genes such as Ccl2, Nfe212 or Mt1 are common for these three cell types, suggesting a possible common activation signature in ALF. Indeed, as many as 82 commonly expressed genes were identified in AAs, AAe and AAk cells (FIG. 4A). A Monte Carlo simulation to estimate the odds of such a commonality using 10⁹ iterations did not observe a single instance of overlap with as many genes, strongly hinting towards a common transcriptional response program underlying these common expression patterns. A search for transcription factor binding site sequence enrichment within the promoters of this gene set yielded multiple different MYC binding motifs, suggesting that it may be a regulator of this response (FIG. 4B). To further corroborate this enrichment, permutation analysis of number of MYC binding sites within 77 randomly chosen genes was performed. 10¹⁰ iterations resulted with a distribution of a mean 197 binding sites and a maximum value of 343 binding sites, while within the common activation signature 402 MYC binding sites were (FIG. 16A). Furthermore, expression of Myc itself also trended towards upregulation in ALF, but did not reach a statistical significance (FIG. 9A). At the protein level, a significant elevation of MYC in mice treated with APAP or TAA in comparison to controls (FIGS. 17 and 16C), while phosphorylated MYC remained unchanged (FIG. 16D).

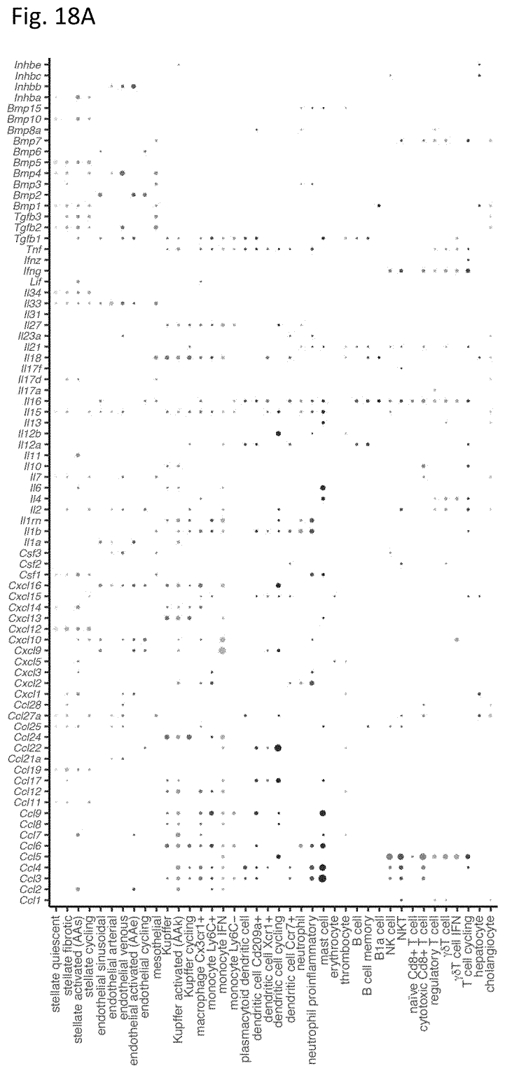

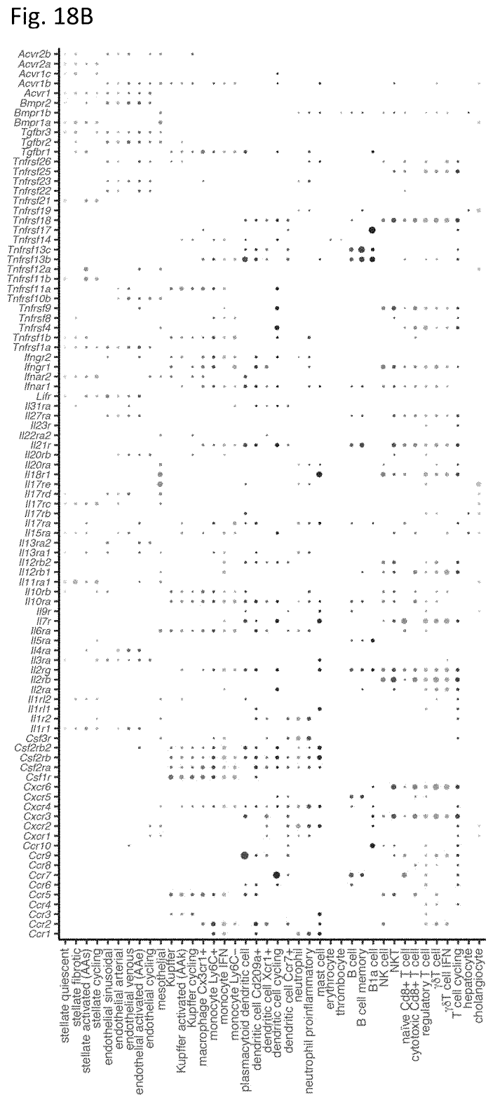

Upon activation, all three resident cell types described above expressed chemokines that target cells for recruitment, hence in the nest step the infiltrating cell characteristics during ALF were investigated. Indeed, in parallel to reduced cell proliferation of resident cells during ALF (FIG. 7A) and the activation phenotype assumed by these resident cell subsets, three populations of expanded hepatic subsets that did not have corresponding quiescent counterparts and thus likely represented infiltrating cells were identified. Ly6C-positive monocytes and Cd209a-positive dendritic cells expressed Ccr2, and Ccl2 was previously reported to be responsible for monocyte recruitment⁴. Interestingly, the number of Xcr1-positive dendritic cells that were also positive for Ccr2 relatively decreased (FIG. 8), most likely as a result of infiltration of large numbers of monocytes and an increase of total number of immune cells. In contrast, the neutrophil infiltrating fraction did not express Ccr2, and was likely recruited via a different mechanism^{35,36}, possibly the highly expressed Ccr1 or Cxcr2 (FIGS. 2M and 18A-B). As both the infiltrating ALF-associated neutrophil and monocyte subsets were heterogeneous, they were further dissected in decoding possible distinct functional roles of their subsets.

Heterogeneity of Neutrophils

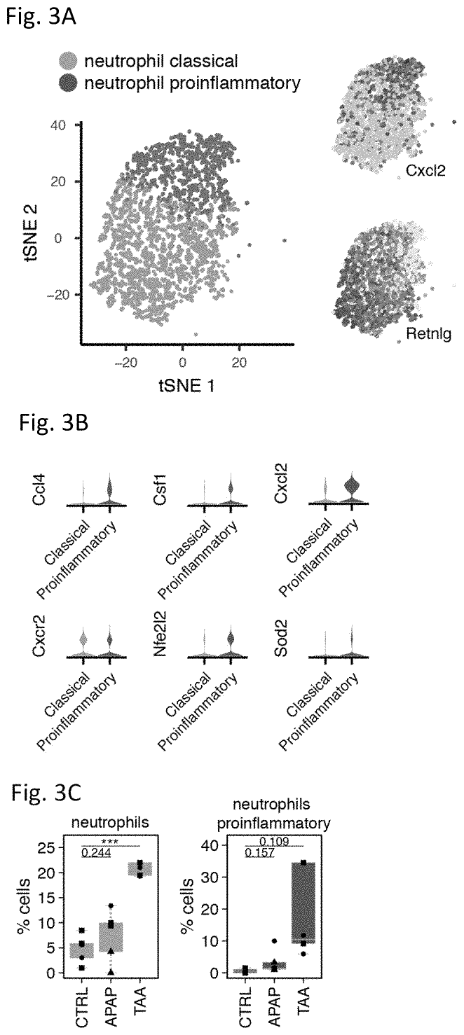

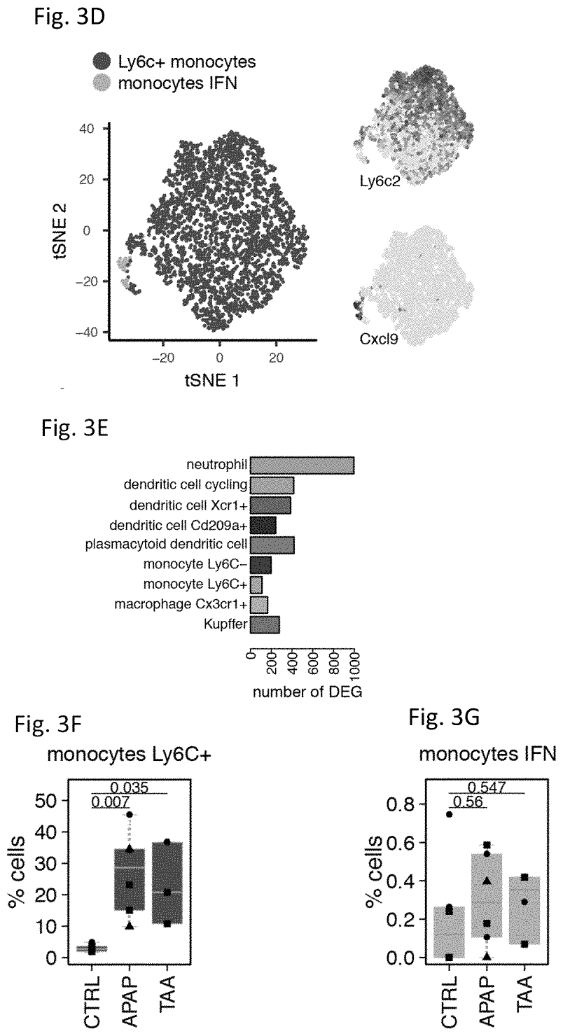

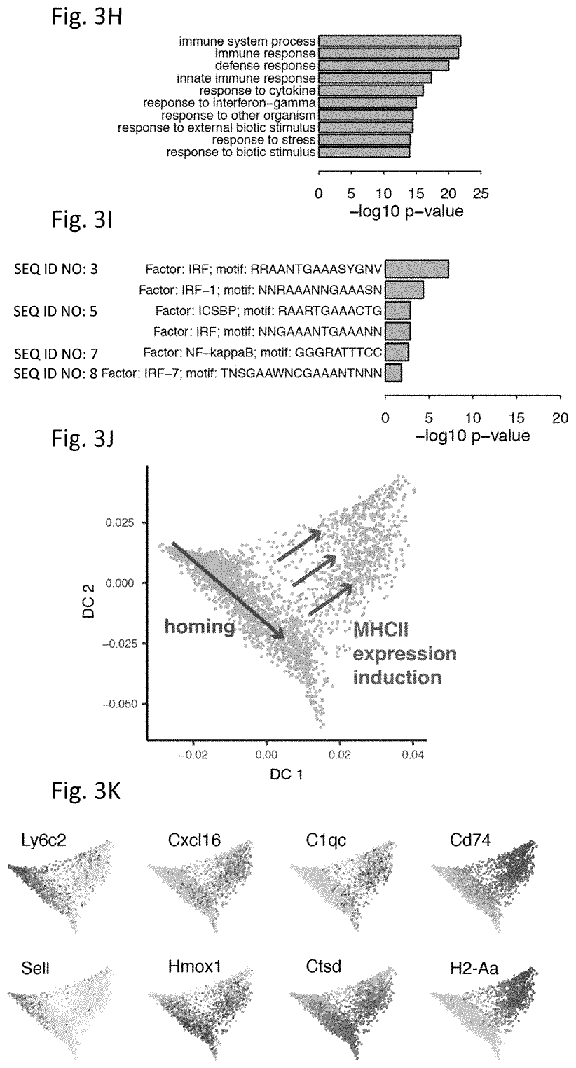

Two neutrophil subpopulations, of 1968 and 1014 cells, respectively, were identified. Differential expression analysis between these two subsets identified 128 differentially expressed genes (FIG. 3A). The larger subset represented classical tissue-resident neutrophils³⁷. The smaller subpopulation expressed Ccl3, Ccl4 and Csf1, which suggests that these cells were likely the previously described pro-inflammatory neutrophil subtype³⁸. These neutrophils also expressed Nfe212 encoding the NRF2 transcription factor known for regulation of antioxidant transcriptional program³⁹. Interestingly, Cxcr2 was downregulated upon neutrophil activation, possibly suggesting that it may be involved in mediating infiltration and downregulated thereafter (FIG. 3B). Neutrophil infiltration and activation were more pronounced in TAA-treated as compared to APAP-treated mice (FIG. 3C).

Heterogeneity of Ly6C-Positive Monocytes

Ly6C-positive monocytes have been suggested to infiltrate the liver in a number of pathologies⁴⁰. In the naïve liver, two populations of Ly6C-positive monocytes were identified: a main population of 3385 cells and a small, but distinct, subpopulation of only 62 cells (FIG. 3D). Differential expression analysis to other innate immune cell subsets revealed that the small subset was most similar to Ly6C monocytes (FIG. 3E). Of 111 differentially-expressed genes between the larger and smaller monocyte subsets, 75 genes were upregulated in the smaller subpopulation. During ALF, a strong infiltration of Ly6C-positive macrophages of the larger subset was observed (FIG. 3F), but the small population percentage remained unchanged (FIG. 3G). Further GO analysis showed that the differentially-expressed genes in the smaller subpopulation were associated with immunity-related terms (FIG. 3H). Analysis of their promoter binding sites showed a striking enrichment of binding motifs for IRF family transcription factors (FIG. 3I), all hallmarks of a response to interferon signaling, hence these cells were named IFN-activated monocytes. Indeed, this subpopulation strongly upregulated Cxcl9 and Cxcl10 chemokines, which were previously shown to be regulated by interferon gamma⁴¹. Of note, interferon itself was detected only in very few T cells, precluding further assessment of interferon secretion in the tested ALF models.

The main population of Ly6C-positive monocytes exhibited further underlying heterogeneity which was decoded using diffusion maps⁴². These revealed that gene expression heterogeneity in this monocyte cluster stems from two processes, one consisting of monocyte homing to the liver and the other induction of MHCII complex genes expression (FIG. 3J). Of note, monocyte homing led to a gradual loss of expression of Ly6C and Sell in monocytes, and gained expression of Cxcl16, C1qa, Hmox1 and cathepsins (FIG. 3K).

Analysis of additional cells yielded the same trend of results further confirming the finding presented herein.

### Example 2

### Inhibition of a Novel TLR-MYC Signaling Pathway Ameliorates Acute Liver Failure

MYC Inhibition Leads to Amelioration of ALF

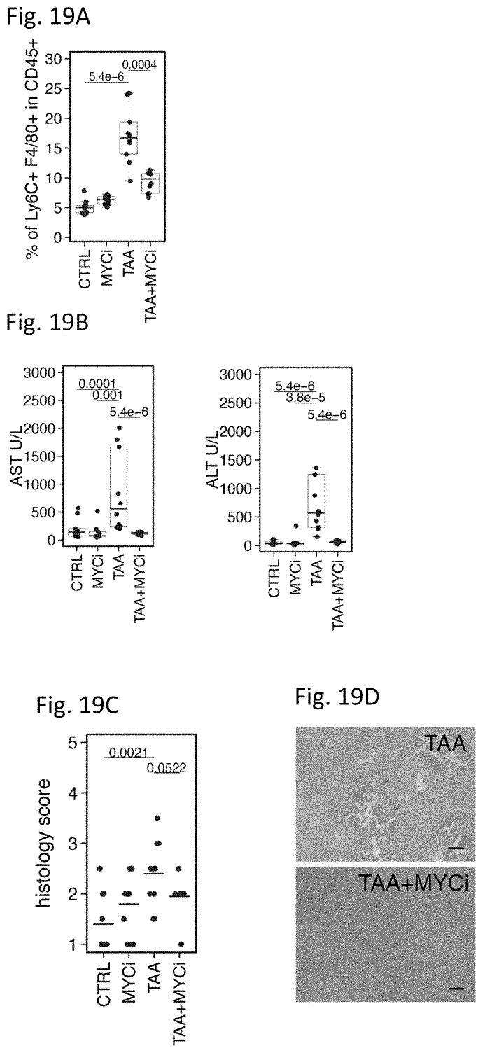

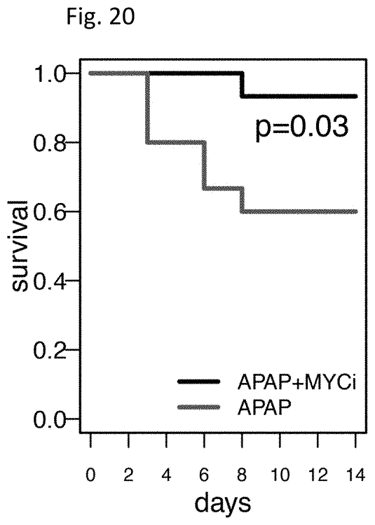

As the search for transcription factor binding site sequence enrichment and expression analysis indicated MYC as a regulator in response to ALF, it was reasoned that MYC activation contributes to the altered stellate, endothelial and Kupffer cell states described above, while MYC inhibition may attenuate resident cellular response to ALF-induced signals through expression of the above common gene-expression signature. Such inhibition, including that of the upregulation of Ccl2, the key chemokine for monocytes recruitment, may also lead to an impairment in Ly6C-positive monocyte infiltration that further contributes to ALF-induced hepatic damage. To test this hypothesis, ALF was induced with APAP and the mice were co-treated with the MYC inhibitor KJ-Pyr-9⁴³ (hereinafter MYCi) and the impact of MYC inhibition on infiltration of Ly6C-positive monocytes, serum levels of ALT and liver histology was evaluated. Monocyte infiltration was assessed by FACS as amount of F4/80 and Ly6C double-positive cells within the immune CD45-positive population (FIG. 9B). Indeed, a dramatic reduction in monocyte infiltration in mice induced with ALF and co-treated with MYCi was observed, suggesting that MYC may play a role in induction of the inflammatory response to liver damage in this setting (FIGS. 4C and 19A). Serum AST and ALT activity in both ALF models (FIGS. 4D-E and 19B) and mortality in APAP-administered mice (FIG. 20) were likewise attenuated upon MYC inhibition. Tissue histology by H&E staining demonstrated that MYC inhibition led to reduced hepatic damage during ALF (FIGS. 4F-G and 19C-D).

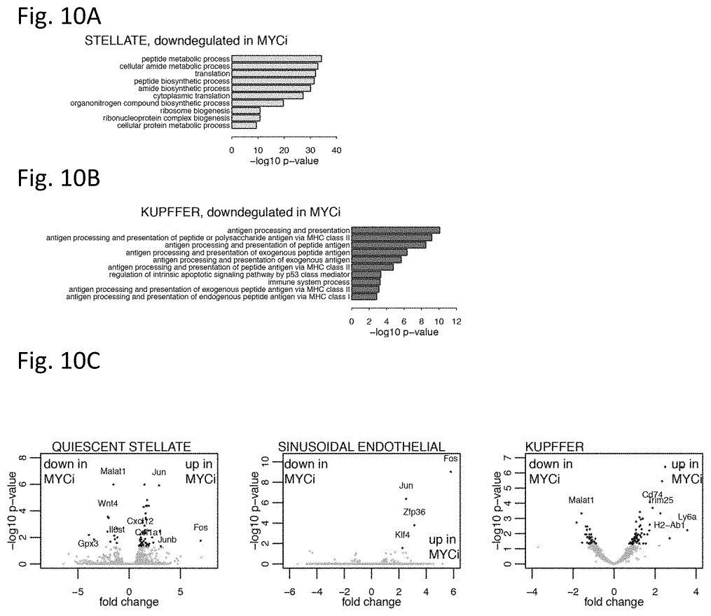

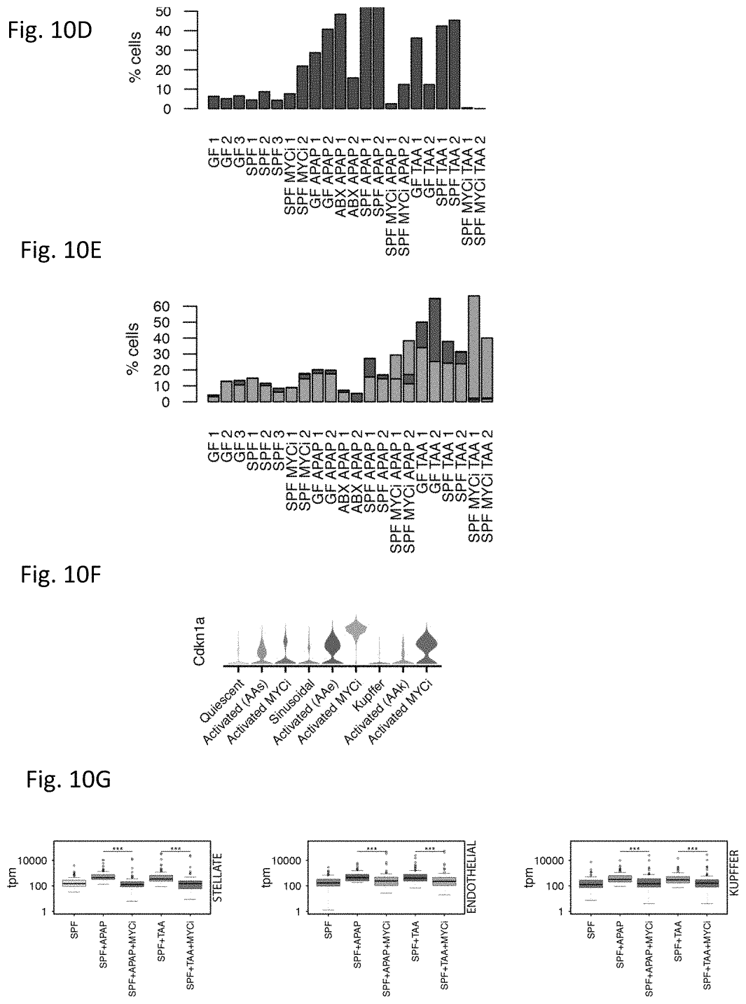

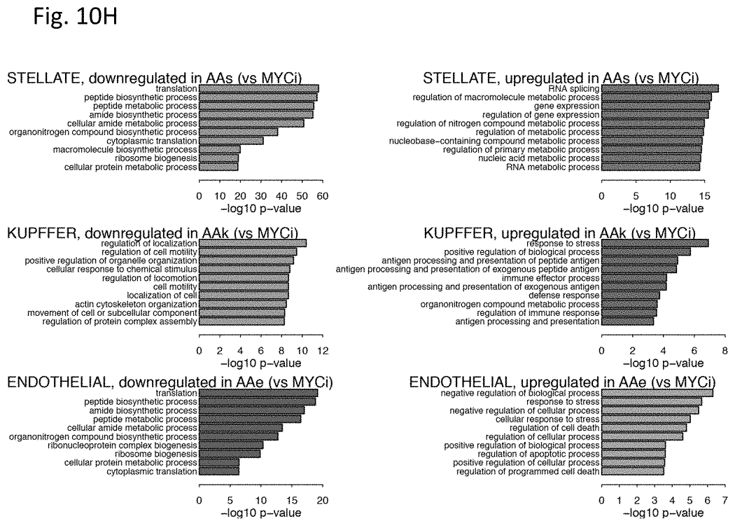

To corroborate these results, single cell RNA sequencing was performed in mice receiving MYCi in the APAP and TAA ALF models, or in the absence of acute hepatic insult, to examine the effect of MYC inhibition on liver cell-specific gene expression patterns. In the absence of liver failure, MYCi did not feature a notable effect on the gene expression landscape (FIGS. 4H and 9C). A closer examination by differential expression analysis of pseudobulk counts between samples revealed that during steady state MYC inhibition led to differential expression of 148 genes in stellate cells, 94 in Kupffer cells and 5 in endothelial cells. Gene ontology analysis of differentially expressed genes in the presence of MYCi in stellate cells revealed downregulation of genes coding for ribosome proteins and other components of the translation machinery, coupled with upregulation of terms related to developmental processes, which included among others, genes from AP1 family (Fos, Jun, Junb) as well as Col3a1 and Cxcl12 (FIG. 10A). Similarly, in endothelial cells, MYC inhibition during steady state induced a downregulation of genes of the AP1 family, while in Kupffer cells MYC inhibition drove a downregulation of genes related to antigen processing and presentation (mainly MHCII genes) (FIGS. 10B-C).

Importantly, during ALF induction in presence of MYCi, activated populations (AAs, AAe, AAk) did not arise, and instead new cellular states of stellate, endothelial, Kupffer, dendritic and T cells were observed; that were markedly different from the populations found in the absence of MYCi and did not cluster together with conventional activation states (FIGS. 4H and 9C). In addition, single cell transcriptomic data recapitulated that, in the presence of MYCi, a near-total reduction in the infiltration of Ly6C-positive monocytes to the liver was noted in either APAP- or TAA-induced ALF (FIG. 10D). Interestingly, neutrophil infiltration was not affected by MYCi (FIG. 10E). A potential explanation to this differential effect on MYC inhibition may involve the neutrophil attractant chemokine Cxcl2, which did not get downregulated by the presence of MYCi, unlike the monocyte chemoattractant Ccl2 (FIG. 41). Importantly, expression of the vast majority of the 82 genes constituting the common activation signature in activated stellate cell, Kupffer cell and endothelial cell subsets was markedly attenuated upon MYCi treatment. Only two genes, metallothionein 1 and 2 (Mt1 and Mt2), remained unaffected by MYC inhibition, suggesting that expression of these oxidative stress response genes is regulated by a different mechanism (FIGS. 4J and 10G).

Interestingly, MYC inhibition in ALF led to a lower total gene expression level. The strongest effect was observed in stellate cells, where the median number of detected transcripts dropped almost 4 times, from 3500 to 945. This effect was not a result of technical differences between samples, as the median number of detected transcripts in other cell types did not differ as much (FIG. 6E). One potential explanation for this reduction is apoptosis, which is associated with rapid mRNA decay and decrease in mitochondrial content⁴⁴. Indeed, a strong downregulation of transcript number, with no increase in percentage of mitochondrial reads suggested that stellate cells undergo cell death in the absence of MYC activity (FIG. 6G). The process seemed to be specific to stellate cells, as endothelial and Kupffer cells did not feature these cell death hallmarks. Moreover, upregulation of senescence and cell cycle arrest marker Cdkn1a (coding p21) in activated cells was attenuated in stellate cells upon MYC inhibition (FIG. 10F). Gene ontology analysis of genes upregulated in endothelial cells activated in the presence of MYC inhibition revealed terms related to apoptosis, its negative regulation and metabolism, while such analysis of Kupffer cells demonstrated mainly changes in immune response-related terms (FIG. 10H).

Microbiome Depletion Leads to Amelioration of ALF

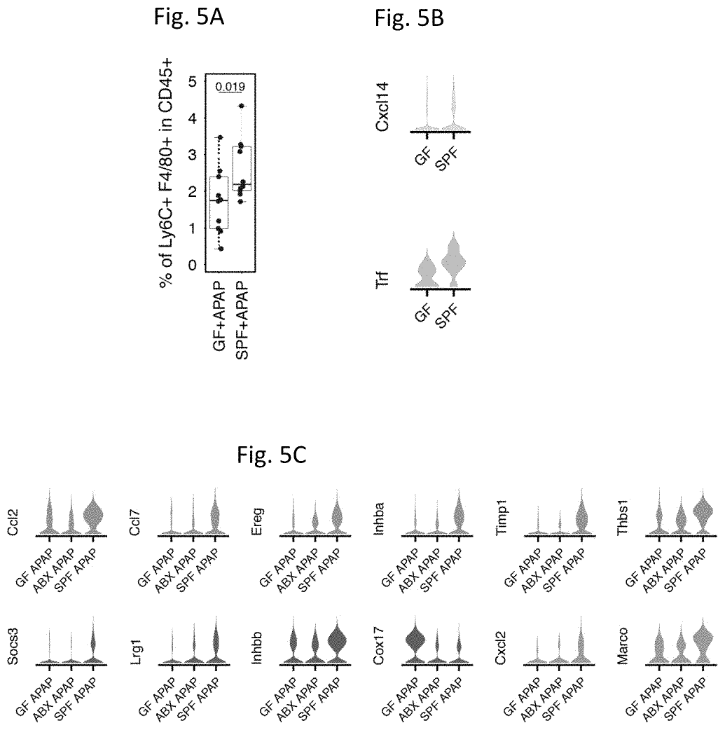

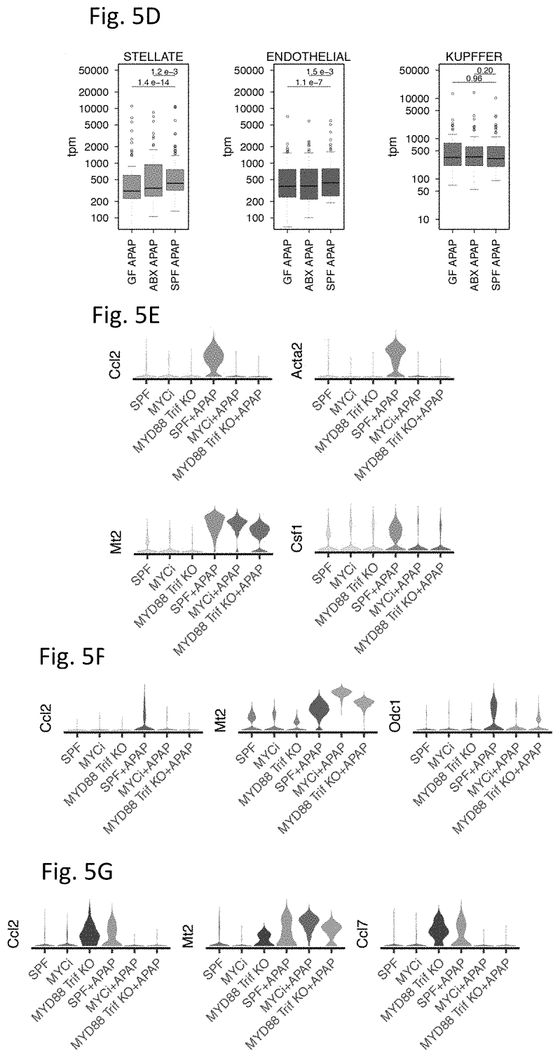

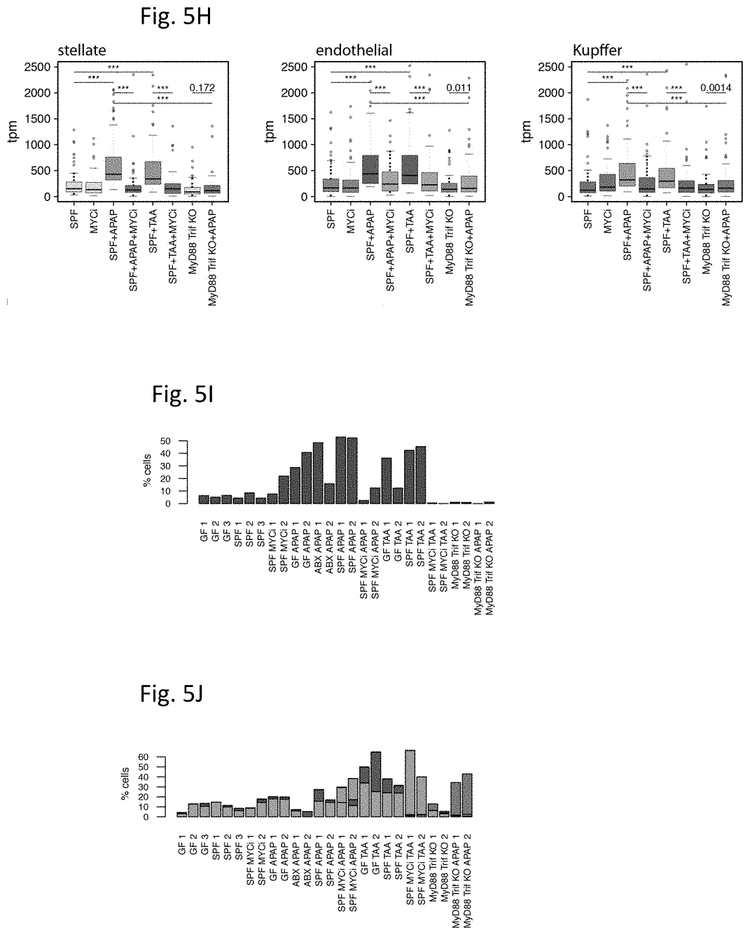

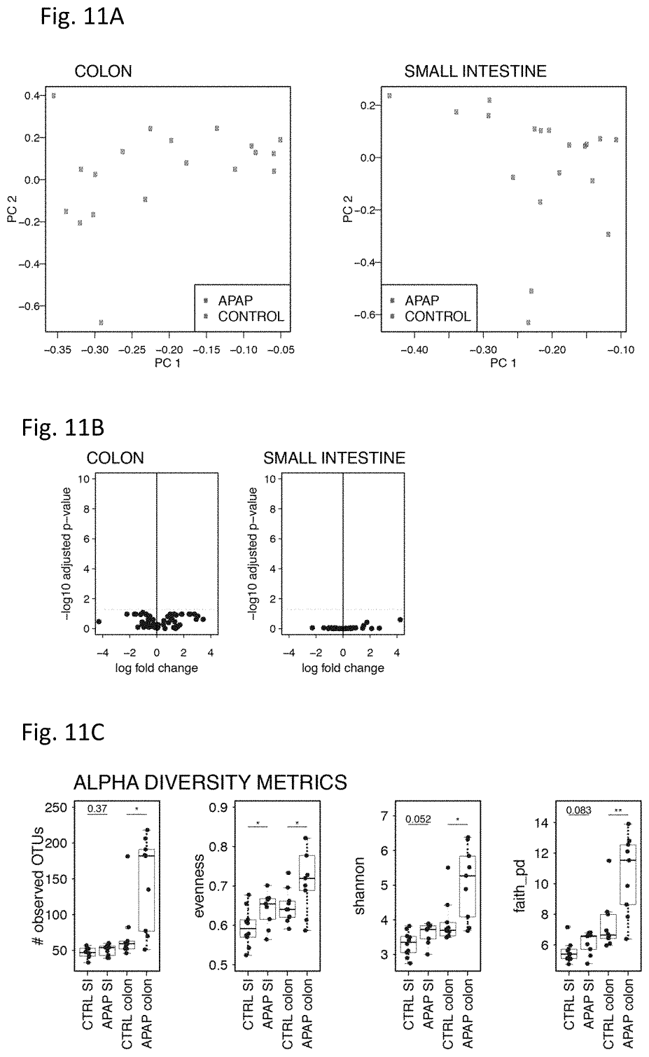

Given the discrepancy between the profound ameliorative effect of microbiome depletion in ALF (FIG. 6C), and the lack of gross differences between GF and SPF resident liver cells in this setup (FIG. 1C), the present inventors sought to elucidate the possible effects of the gut microbiome on more subtle ALF-induced cell-specific gene expression programs. First, differences in microbiome composition associated with ALF were searched for by performing a 16S gene V4 region amplicon sequencing of colon and jejunum content during disease induction. No major differences were found in relative abundance, other than an increase in alpha diversity in ALF (FIG. 11A-C). Importantly, while resident cell subsets did not differ during ALF between microbiome-depleted or GF mice to colonized mice (FIGS. 1A-D), APAP-induced ALF was associated with a significantly enhanced infiltration of Ly6C monocytes in SPF mice in comparison to GF mice (FIG. 5A), in agreement with the attenuated APAP-induced liver toxicity noted in GF mice (FIG. 6C)⁴⁵.

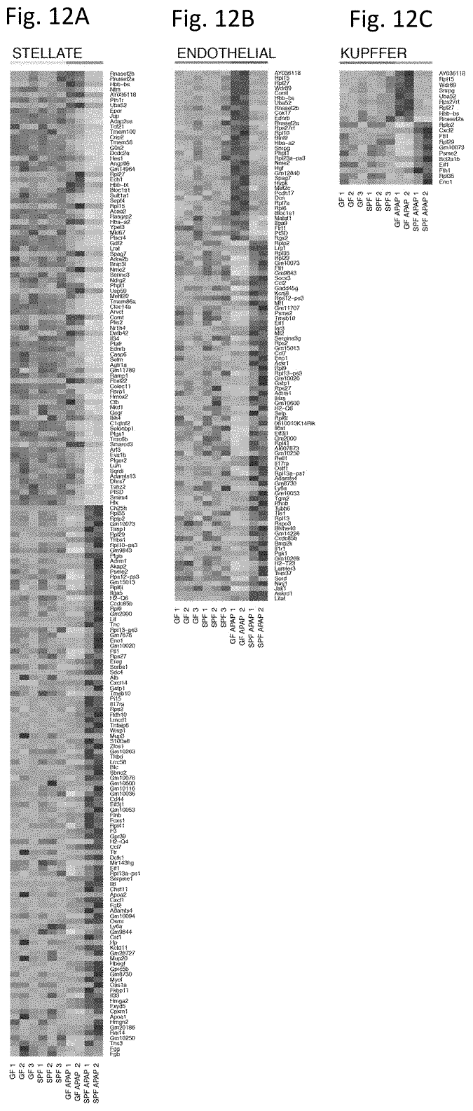

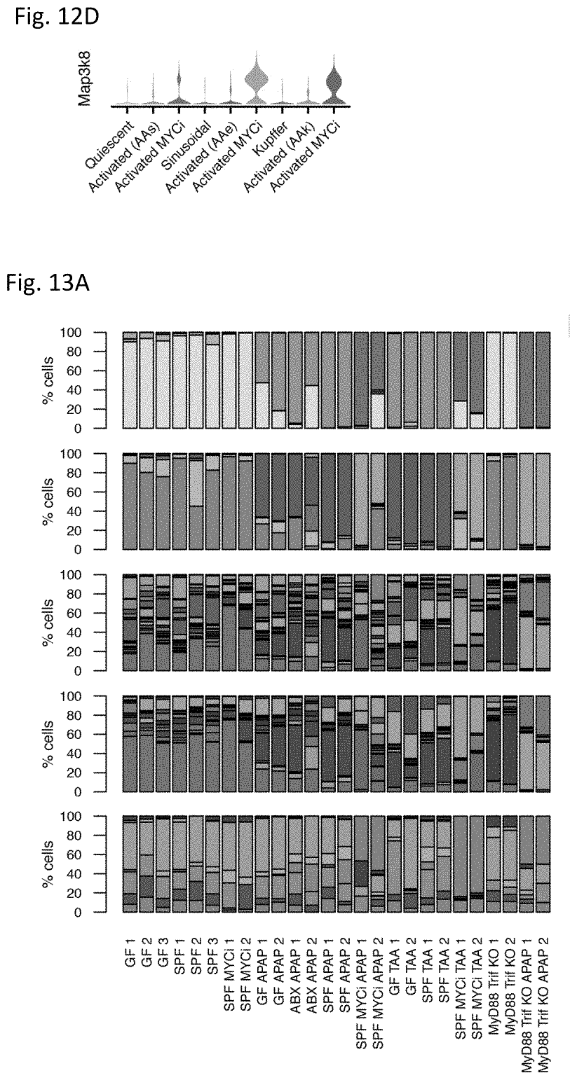

To determine whether the functional differences in hepatic resident cells contributed to this enhanced microbiome-induced monocyte infiltration, a differential expression between GF and SPF mice using pseudobulk counts was performed for each cell type with DESeq2. In naïve mice, the analysis revealed only two differentially expressed genes between SPF and GF mice—Cxcl14 in stellate cells and Tfr in cholangiocytes (DESeq2 FDR adjusted p-value 3.73·10⁻⁶ and 1.97·10⁻⁶ respectively, FIG. 5B)⁴⁶. In contrast, comparison of differential gene expression between APAP-administered SPF and GF mice revealed 186 differentially expressed genes between AAs cells, 106 in AAe cells and 20 in AAk cells. No differentially abundant genes were found between AAs, AAe and AAk cells in GF and SPF mice in the TAA model. Among the more abundant genes in APAP ALF-induced SPF mice, was Cxcl1, Cxcl14 and Csf1 in stellate cells, Ccl2 and Ccl7 in endothelial cells and Cxcl2 in Kupffer cells, which collectively may contribute to the difference in the infiltration of Ly6C-positive monocytes between SPF and GF mice during ALF (FIG. 5C). Moreover, interleukin 6 family members (Il6, Lif) and activation markers such as Thbs1, Timp1, Cd44, Itga5, Il17ra and Ereg featured higher expression levels in activated stellate cells in SPF mice, in agreement with previously shown dependence of Ereg expression on microbiome via TLR4 signaling in hepatocellular carcinoma mouse model. In SPF-activated endothelial cells, higher levels of activation markers such as Lrg1, Rspo3, Mt1, Mt2 and Bhlhe40 were observed, as compared to GF mice. Many stellate cell and endothelial cell genes upregulated in SPF mice were related to the translation machinery. In Kupffer cells, 11 genes were found to be upregulated in SPF mice, including Eno1 and Cxcl2. Only a few genes in germ-free mice were expressed in higher levels than in SPF mice and Rnaset2a and Rnaset2b in stellate and endothelial cells (FIGS. 5C and 12A-C). Depletion of the microbiome with antibiotics, led to similar gene expression changes that were intermediate in their extent between those of GF and SPF conditions.

These noted microbiome gene-expression effects raised the possibility that the entire MYC-regulated ALF signature may be affected by the microbiome. Indeed, the mean expression of the MYC-regulated gene signature in stellate and endothelial cells was higher in SPF than in GF mice, while ABX-treatment induced an intermediate expression level between these two extreme colonization conditions (FIG. 5D). Together, these results suggest that microbiome-mediated upstream signals may regulate MYC during ALF.

TLR Signaling Inhibition Leads to Amelioration of ALF

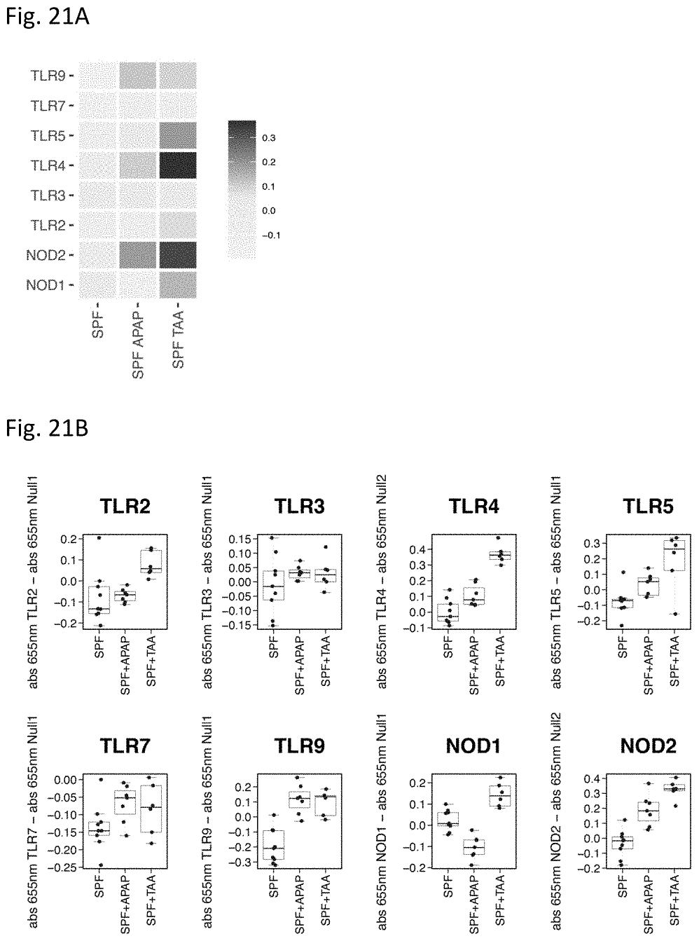

It was hypothesized that the microbiome regulates the MYC program in hepatic stellate, endothelial, and Kupffer cells through TLR signaling. In this scenario, signaling by damage associated molecular patterns (DAMPs) originating from damaged liver cells, coupled with portal venous PAMPs originating from the gut microbiome jointly drive a TLR-induced MYC activation in these cells, driving downstream immune cell infiltration and exacerbated disease. Indeed, a reporter cell assay (methods) identified portal vein TLR2, TLR4, TLR5, TLR9, NOD1, and NOD2 agonists upon induction of TAA ALF, and TLR4, TLR9 and NOD2 agonists upon induction of APAP ALF (FIGS. 21A-B). To test whether a possible TLR involvement exists downstream of these microbial-associated molecular patterns (MAMPs), MyD88-Trif double knockout (MyD88-Trif dKO) mice, which lack both adaptor proteins necessary for TLR signaling were utilized and single cell RNAseq was performed at both naïve and APAP-treated conditions in the MyD88-Trif dKO mice and in wildtype (WT) littermate controls.

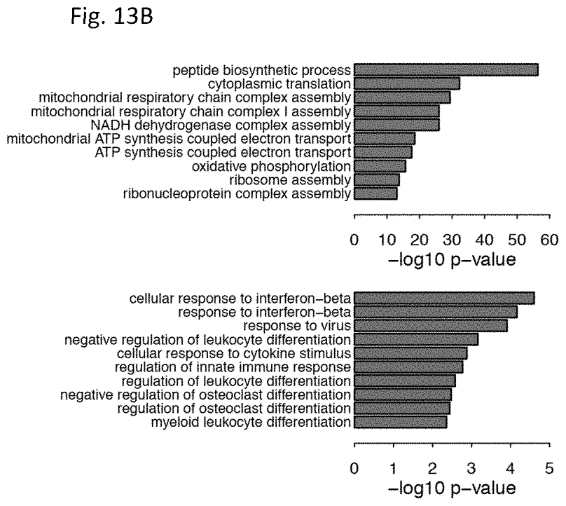

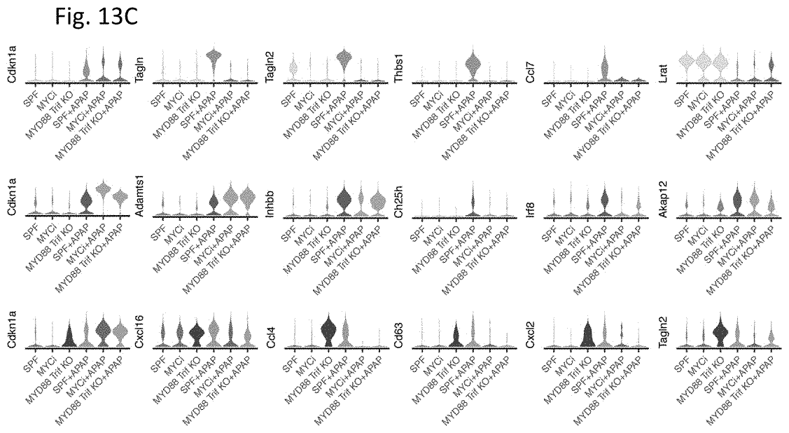

In steady state, all cellular states in MyD88-Trif dKO mice were similar to WT mice, except for MyD88-Trif dKO Kupffer cells which clustered separately from the respective cells in WT mice (FIG. 13A). Interestingly, MyD88-Trif dKO Kupffer cells featured higher expression of interferon responsive factors as compared to WT Kupffer cells (FIG. 13B).

Importantly, upon induction of APAP-driven ALF, MyD88-Trif dKO stellate and endothelial cells became aberrantly activated, assuming a transcriptional state distinct from that of ALF-induced WT mice, and nearly identical to that of MYCi-treated mice (FIGS. 9C and 13A). MyD88-Trif dKO Kupffer cells also became aberrantly activated, but their activation state was distinct from that of both APAP-induced and MYCi-treated APAP-induced WT mice. Similarly, neutrophils assumed an activate pattern markedly different from that observed in ALF-induced WT controls (FIGS. 9C and 13A).

In corroboration of these findings, stellate, endothelial and Kupffer cells in MyD88 Trif dKO mice expressed methalothioneins Mt1 and Mt2 in response to APAP, but failed to express Ccl2, Ccl7, Acta2, Csf1, Inhbb, similarly to MYCi-treated mice in which Myc transcriptional activity was inhibited (FIGS. 5E-G and 13C). Furthermore, expression of the ‘signature’ 82-gene MYC-induced pattern in stellate, endothelial and Kupffer cells was significantly attenuated in MyD88-Trif dKO mice as compared to WT littermate controls, similarly to what was observed upon MYCi treatment (FIG. 5G). Moreover, ALF-induced monocyte infiltration was blocked in the absence of TLR signaling in MyD88-Trif dKO mice, while that of neutrophils remained unaffected. These results suggest that the ALF MYC program in stellate, endothelial and Kupffer cells is regulated via upstream TLR signaling, while microbiome depletion or TLR inhibition induces a marked suppression of this cell-specific MYC program, thereby driving a significant attenuation of ALF.

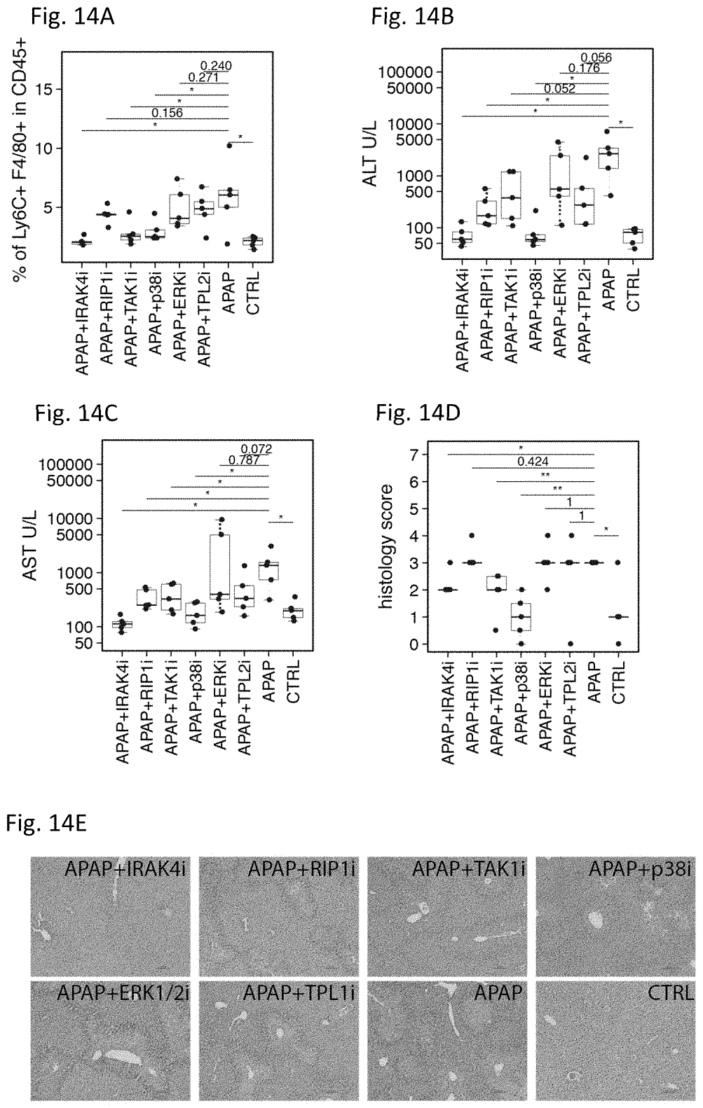

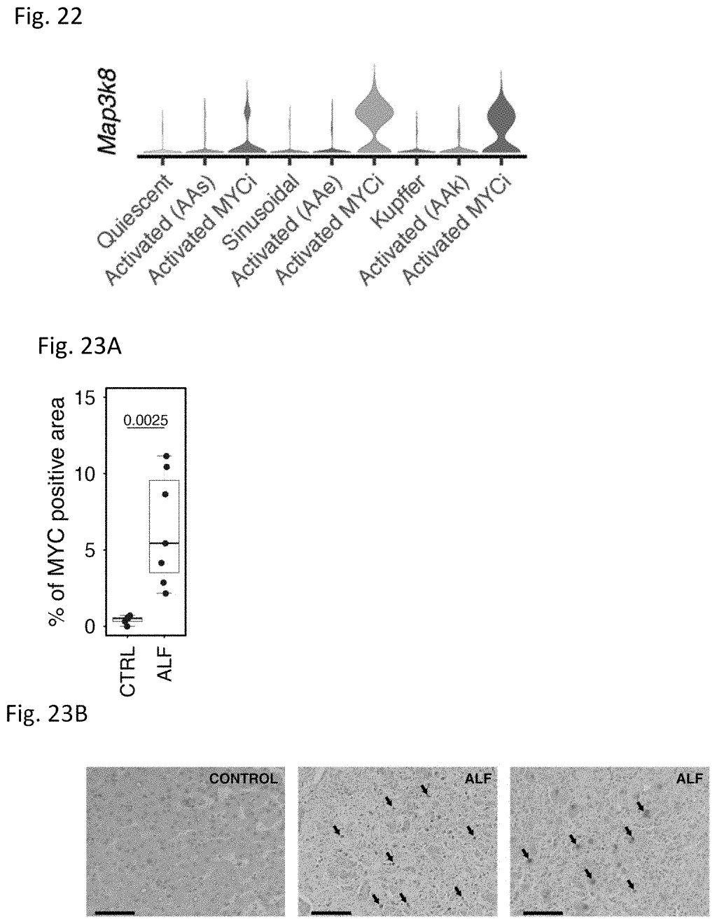

Given this suggestive TLR-MYC signaling pathway involvement in ALF, the effect of inhibiting components along the pathway has been evaluated. One apparent candidate pathway is the MAPK pathway, relaying signals from TLRs sensing MAMPs and DAMPs to regulate MYC-dependent gene expression. In support of such pathway involvement in ALF are observations suggesting that TLR4 signaling regulates microbiota-dependent Ereg expression in hepatocellular carcinoma in stellate cells [Clayton, T. A., et al. Proc. Natl. Acad. Sci. 106, 14728-14733 (2009)], the senescence-associated secretory phenotype being downregulated in absence of TLR2 [Hari, P. et al. Sci Adv 5, eaaw0254 (2019)], and the strong induction of Map3k8 expression (coding for TPL2) noted in the presence of MYCi during ALF (FIG. 22). Hence, to test the MAPK pathway involvement in ALF six proteins from the pathway were selected; namely, inhibition of IRAK4, RIP1, TAK1, TPL2, ERK1/2 or p38, was tested in APAP-induced ALF model (Table 1 hereinabove and FIGS. 14A-E). Indeed, a significant reduction of monocyte infiltration was observed in mice receiving inhibitors of IRAK4, TAK1 and p38 (FIG. 14A). AST activity in serum was also significantly reduced in the presence of IRAK4, RIP1, TAK1 and p38 inhibitors; and ALT activity was significantly reduced in the presence of IRAK4, RIP1 and p38 inhibitors (FIGS. 14B-C). Histopathological analysis recapitulated these results, demonstrating a significantly reduced liver damage in mice subjected to IRAK4, TAK1 or p38 inhibition (FIG. 14D).

### Example 3

### MYC is Upregulated in Human ALF

In the next step, the present inventors evaluated whether the noted MYC involvement in animal models of ALF may be observed in human patients. To this end, the levels of MYC were quantified by immunohistochemistry in hepatic liver sections obtained from 7 ALF patients (Table 2 hereinbelow. As healthy controls, liver samples obtained from 5 healthy liver donors were used. Indeed, a significant increase in nuclear MYC protein levels was noted in ALF patients as compared to controls (FIG. 23A-B).

TABLE 2
Details of human liver samples including age in years,
gender (F = female, M = male) and disease status.

| Sample | Age | Gender | Etiology |
|---|---|---|---|
| 19-05774 | 72 | F | Liver donor |
| 19-12173 | 77 | M | Liver donor |
| 18-20437 | 32 | F | Liver donor |
| 18-20858 | 64 | M | Liver donor |
| 18-10530 | 63 | M | Liver donor |
| 12-09634 | 47 | M | Fulminant hepatic failure undetermined |
| | | | etiology |
| 14-2115 | 6 | M | Fulminant hepatic failure undetermined |
| | | | etiology |
| 15-02328 | 12 | F | Fulminant hepatic failure undetermined |
| | | | etiology |
| 18-13822 | 72 | M | Fulminant hepatic failure with autoimmune |
| | | | features |
| 18-13906 | 81 | F | Fulminant hepatic failure with autoimmune |
| | | | features |
| 19-16184 | 2 | M | Fulminant hepatic failure undetermined |
| | | | etiology |
| 19-11747 | 2 | F | Fulminant hepatic failure undetermined |
| | | | etiology |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Bernal, W. & Wendon, J. Acute liver failure. N. Engl. J. Med. 369, 2525-2534 (2013).
2. Hinson, J. A., Roberts, D. W. & James, L. P. Mechanisms of acetaminophen-induced liver necrosis. Handb. Exp. Pharmacol. 369-405 (2010).
3. Jaeschke, H. Reactive oxygen and mechanisms of inflammatory liver injury: Present concepts. J. Gastroenterol. Hepatol. 26 Suppl 1, 173-179 (2011).
4. Dambach, D. M., Watson, L. M., Gray, K. R., Durham, S. K. & Laskin, D. L. Role of CCR2 in macrophage migration into the liver during acetaminophen-induced hepatotoxicity in the mouse. Hepatology 35, 1093-1103 (2002).
5. Mossanen, J. C. et al. Chemokine (C-C motif) receptor 2-positive monocytes aggravate the early phase of acetaminophen-induced acute liver injury. Hepatology 64, 1667-1682 (2016).
6. Jaeschke, H., Williams, C. D., Ramachandran, A. & Bajt, M. L. Acetaminophen hepatotoxicity and repair: the role of sterile inflammation and innate immunity. Liver Int. 32, 8-20 (2012).
7. Dapito, D. H. et al. Promotion of hepatocellular carcinoma by the intestinal microbiota and TLR4. Cancer Cell 21, 504-516 (2012).
8. Henao-Mejia, J. et al. Inflammasome-mediated dysbiosis regulates progression of NAFLD and obesity. Nature 482, 179-185 (2012).
9. Llopis, M. et al. Intestinal microbiota contributes to individual susceptibility to alcoholic liver disease. Gut 65, 830-839 (2016).
10. Kolodziejczyk, A. A., Kim, J. K., Svensson, V., Marioni, J. C. & Teichmann, S. A. The technology and biology of single-cell RNA sequencing. Mol. Cell 58, 610-620 (2015).
11. Hajovsky, H. et al. Metabolism and Toxicity of Thioacetamide and Thioacetamide S-Oxide in Rat Hepatocytes. Chemical Research in Toxicology 25, 1955-1963 (2012).
12. Woolbright, B. et al. The impact of sterile inflammation in acute liver injury. Journal of Clinical and Translational Research (2017). doi:10.18053/jctres 0.03.2017s1.003
13. Elinav, E. et al. NLRP6 inflammasome regulates colonic microbial ecology and risk for colitis. Cell 145, 745-757 (2011).
14. Mederacke, I., Dapito, D. H., Affò, S., Uchinami, H. & Schwabe, R. F. High-yield and high-purity isolation of hepatic stellate cells from normal and fibrotic mouse livers. Nat. Protoc. 10, 305-315 (2015).
15. Heng, T. S. P., Painter, M. W. & Immunological Genome Project Consortium. The Immunological Genome Project: networks of gene expression in immune cells. Nat. Immunol. 9, 1091-1094 (2008).
16. Strauss, O., Phillips, A., Ruggiero, K., Bartlett, A. & Dunbar, P. R Immunofluorescence identifies distinct subsets of endothelial cells in the human liver. Sci. Rep. 7, 44356 (2017).
17. Guilliams, M. et al. Unsupervised High-Dimensional Analysis Aligns Dendritic Cells across Tissues and Species. Immunity 45, 669-684 (2016).
18. Ohl, L. et al. CCR7 governs skin dendritic cell migration under inflammatory and steady-state conditions. Immunity 21, 279-288 (2004).
19. Zilionis, R. et al. Single-Cell Transcriptomics of Human and Mouse Lung Cancers Reveals Conserved Myeloid Populations across Individuals and Species. Immunity 50, 1317-1334.e10 (2019).
20. Yang, C.-Y. et al. CLEC4F is an inducible C-type lectin in F4/80-positive cells and is involved in alpha-galactosylceramide presentation in liver. PLoS One 8, e65070 (2013).
21. Krenkel, O. & Tacke, F. Liver macrophages in tissue homeostasis and disease. Nat. Rev. Immunol. 17, 306-321 (2017).
22. MacParland, S. A. et al. Single cell RNA sequencing of human liver reveals distinct intrahepatic macrophage populations. Nat. Commun. 9, 4383 (2018).
23. Krenkel, O., Hundertmark, J., Ritz, T. P., Weiskirchen, R. & Tacke, F. Single Cell RNA Sequencing Identifies Subsets of Hepatic Stellate Cells and Myofibroblasts in Liver Fibrosis. Cells 8, (2019).
24. Tsuchida, T. & Friedman, S. L. Mechanisms of hepatic stellate cell activation. Nat. Rev. Gastroenterol. Hepatol. 14, 397-411 (2017).
25. Reynaert, H., Thompson, M. G., Thomas, T. & Geerts, A. Hepatic stellate cells: role in microcirculation and pathophysiology of portal hypertension. Gut 50, 571-581 (2002).
26. Schmidt-Arras, D. & Rose-John, S. IL-6 pathway in the liver: From physiopathology to therapy. J. Hepatol. 64, 1403-1415 (2016).
27. Maeshima, K. et al. A protective role of interleukin 11 on hepatic injury in acute endotoxemia. Shock 21, 134-138 (2004).
28. Campisi, J. & d'Adda di Fagagna, F. Cellular senescence: when bad things happen to good cells. Nat. Rev. Mol. Cell Biol. 8, 729-740 (2007).
29. Coppé, J.-P., Desprez, P.-Y., Krtolica, A. & Campisi, J. The senescence-associated secretory phenotype: the dark side of tumor suppression. Annu. Rev. Pathol. 5, 99-118 (2010).
30. Poisson, J. et al. Liver sinusoidal endothelial cells: Physiology and role in liver diseases. J. Hepatol. 66, 212-227 (2017).
31. Hu, J. et al. Endothelial cell-derived angiopoietin-2 controls liver regeneration as a spatiotemporal rheostat. Science 343, 416-419 (2014).
32. Ding, B.-S. et al. Inductive angiocrine signals from sinusoidal endothelium are required for liver regeneration. Nature 468, 310-315 (2010).
33. Shaulian, E. & Karin, M. AP-1 as a regulator of cell life and death. Nat. Cell Biol. 4, E131-6 (2002).
34. Ramilowski, J. A. et al. A draft network of ligand-receptor-mediated multicellular signalling in human. Nat. Commun. 6, 7866 (2015).
35. Huebener, P. et al. The HMGB1/RAGE axis triggers neutrophil-mediated injury amplification following necrosis. J. Clin. Invest. 125, 539-550 (2019).
36. McDonald, B. et al. Intravascular danger signals guide neutrophils to sites of sterile inflammation. Science 330, 362-366 (2010).
37. Silvestre-Roig, C., Hidalgo, A. & Soehnlein, O. Neutrophil heterogeneity: implications for homeostasis and pathogenesis. Blood 127, 2173-2181 (2016).
38. Mayadas, T. N., Cullere, X. & Lowell, C. A. The multifaceted functions of neutrophils. Annu. Rev. Pathol. 9, 181-218 (2014).
39. Nguyen, T., Nioi, P. & Pickett, C. B. The Nrf2-antioxidant response element signaling pathway and its activation by oxidative stress. J. Biol. Chem. 284, 13291-13295 (2009).
40. Brempelis, K. J. & Crispe, I. N. Infiltrating monocytes in liver injury and repair. Clin Transl Immunology 5, e113 (2016).
41. Groom, J. R. & Luster, A. D. CXCR3 ligands: redundant, collaborative and antagonistic functions. Immunol. Cell Biol. 89, 207-215 (2011).
42. Angerer, P. et al. destiny: diffusion maps for large-scale single-cell data in R. Bioinformatics 32, 1241-1243 (2016).
43. Hart, J. R. et al. Inhibitor of MYC identified in a Kröhnke pyridine library. Proc. Natl. Acad. Sci. U.S.A. 111, 12556-12561 (2014).
44. Thomas, M. P. et al. Apoptosis Triggers Specific, Rapid, and Global mRNA Decay with 3′ Uridylated Intermediates Degraded by DIS3L2. Cell Rep. 11, 1079-1089 (2015).
45. Thaiss, C. A. et al. Microbiota Diurnal Rhythmicity Programs Host Transcriptome Oscillations. Cell 167, 1495-1510.e12 (2016).
46. Maerki, C. et al. Potent and broad-spectrum antimicrobial activity of CXCL14 suggests an immediate role in skin infections. J. Immunol. 182, 507-514 (2009).
47. Yang, L. & Seki, E. Toll-like receptors in liver fibrosis: cellular crosstalk and mechanisms. Front. Physiol. 3, 138 (2012).
48. Seki, E. et al. TLR4 enhances TGF-beta signaling and hepatic fibrosis. Nat. Med. 13, 1324-1332 (2007).
49. Miura, K. et al. Toll-like receptor 2 and palmitic acid cooperatively contribute to the development of nonalcoholic steatohepatitis through inflammasome activation in mice. Hepatology 57, 577-589 (2013).
50. Miura, K. et al. Toll-like receptor 9 promotes steatohepatitis by induction of interleukin-1beta in mice. Gastroenterology 139, 323-34.e7 (2010).
51. Scaffidi, P., Misteli, T. & Bianchi, M. E. Release of chromatin protein HMGB1 by necrotic cells triggers inflammation. Nature 418, 191-195 (2002).
52. Kubes, P. & Mehal, W. Z. Sterile inflammation in the liver. Gastroenterology 143, 1158-1172 (2012).
53. Vénéreau, E., Ceriotti, C. & Bianchi, M. E. DAMPs from Cell Death to New Life. Front. Immunol. 6, 422 (2015).
54. Heard, K. J. Acetylcysteine for acetaminophen poisoning. N. Engl. J. Med. 359, 285-292 (2008).
55. Yamamoto, M. Role of Adaptor TRIF in the MyD88-Independent Toll-Like Receptor Signaling Pathway. Science 301, 640-643 (2003).
56. Stuart, T. et al. Comprehensive Integration of Single-Cell Data. Cell 177, 1888-1902.e21 (2019).
57. Raudvere, U. et al. g:Profiler: a web server for functional enrichment analysis and conversions of gene lists (2019 update). Nucleic Acids Research (2019). doi:10.1093/nar/gkz369
58. Love, M. I., Huber, W. & Anders, S. Moderated estimation of fold change and dispersion for RNA-seq data with DESeq2. Genome Biol. 15, 550 (2014).
59. Bolyen, E. et al. QIIME 2: Reproducible, interactive, scalable, and extensible microbiome data science. (Peer) Preprints, 2018). doi:10.7287/peerj.preprints.27295v2

What is claimed is:

1. A method of treating acute liver disease in a subject in need thereof, the method comprising administering to the subject a therapeutically effective amount of an agent, wherein said agent is:
(a) capable of binding a component of a TLR-MYC signaling pathway selected from the group consisting of MYC, MYD88, TRIF and p38 and inhibiting expression and/or activity of said component, wherein when said component comprises p38 said agent inhibits activity and not expression of said p38 and wherein said acute liver disease is not caused by a hepatitis C virus;
(b) capable of binding a component of a TLR-MYC signaling pathway selected from the group consisting of MYC, MYD88 and TRIF and inhibiting expression and/or activity of said component;
(c) capable of at least one of:
(i) binding a TLR selected from the group consisting of TLR1, TLR2, TLR3, TLR5, TLR6, TLR8 and TLR10 and inhibiting expression and/or activity of said TLR; and/or
(ii) binding at least two different TLRs and inhibiting expression and/or activity of said at least two TLRs, wherein when said TLRs are TLR7 and TLR9, said at least two is at least three; or
(d) capable of binding at least two components of a TLR-MYC signaling pathway and inhibiting expression and/or activity of said at least two components,
thereby treating the acute liver disease in the subject.

2. A method of treating acute liver disease in a subject in need thereof, the method comprising administering to the subject a therapeutically effective amount of an antibiotic, wherein said antibiotic or said therapeutically effective amount inhibits TLR-MYC signaling in liver cells of said subject selected from the group consisting of stellate cells, endothelial cells and Kupffer cells, thereby treating the acute liver disease in the subject.

3. The method of claim 2, further comprising administering to said subject a therapeutically effective amount of an agent capable of binding a component of a TLR-MYC signaling pathway and inhibiting expression and/or activity of said component.

4. The method of claim 3, wherein said component is selected from the group consisting of MYC, TLR, MYD88, IRAK4, TAK1 and p38.

5. The method of claim 3, wherein said TLR is not TLR4.

6. The method of claim 3, wherein said component is selected from the group consisting of MYC, MYD88, IRAK4, TAK1 and p38.

7. The method of claim 1, wherein said at least two components of (d) are selected from the group consisting of MYC, TLR, MYD88, TRIF, IRAK4, TAK1 and p38.

8. The method of claim 2, wherein said antibiotic is a broad spectrum antibiotic.

9. The method of claim 8, wherein said antibiotic is capable of depleting a predominant portion of gut microbiome.

10. The method of claim 1, wherein said agent is a small molecule.

11. The method of claim 1, wherein said agent is an antibody.

12. The method of claim 1, wherein said agent is an RNA silencing agent.

13. A method of treating acute liver disease in a subject in a subject in need thereof, the method comprising administering to the subject a therapeutically effective amount of a toxic agent attached to a targeting moiety for specifically targeting a cell selected from the group consisting of a stellate cell, an endothelial cell and a Kupffer cell, thereby treating the acute liver disease in the subject.

14. The method of claim 13, wherein said targeting moiety is an antibody.

15. The method of claim 1, wherein said acute liver disease is acute liver failure.

16. The method of claim 1, wherein said acute liver disease is a drug-induced acute liver disease.

17. The method of claim 16, wherein said drug is acetaminophen (APAP) or thioacetamide (TAA).

18. The method of claim 1, wherein said acute liver disease is caused by a virus.

19. The method of claim 18, wherein said virus is Hepatitis A virus or Hepatitis B virus.

20. The method of claim 1, wherein said acute liver disease is not caused by a virus.

21. The method of claim 1, wherein said acute liver disease is not caused by a hepatitis C virus.


SEQUENCE LISTING (28 sequences)

SEQ ID NO: 1 - DNA, 63 bp, synthetic construct
  misc_feature 1..63: note = Single strand DNA oligonucleotide
  source 1..63: mol_type = other DNA
aatgatacggcgaccaccgagatctacacgcttatggtaattgtgtgccagcmgccgcggtaa

SEQ ID NO: 2 - DNA, 64 bp, synthetic construct
  misc_feature 1..64: note = Single strand DNA oligonucleotide
  misc_feature 37..44: note = n is a, c, g, or t(barcoding sequence)
  misc_feature 52: note = n is a, c, g, or t
  source 1..64: mol_type = other DNA
caagcagaagacggcatacgagatagtcagccagccnnnnnnnnggactacnvgggtwtctaat

SEQ ID NO: 3 - DNA, 15 bp, synthetic construct
  misc_feature 1..15: note = Transcription factor binding site
  misc_feature 5: note = n is a, c, g, or t
  misc_feature 14: note = n is a, c, g, or t
  source 1..15: mol_type = other DNA
rraantgaaasygnv

SEQ ID NO: 4 - skipped

SEQ ID NO: 5 - DNA, 13 bp, synthetic construct
  misc_feature 1..13: note = Transcription factor binding site
  source 1..13: mol_type = other DNA
rraartgaaactg

SEQ ID NO: 6 - skipped

SEQ ID NO: 7 - DNA, 10 bp, synthetic construct
  misc_feature 1..10: note = Transcription factor binding site
  source 1..10: mol_type = other DNA
gggratttcc

SEQ ID NO: 8 - DNA, 18 bp, synthetic construct
  misc_feature 1..18: note = Transcription factor binding site
  misc_feature 2: note = n is a, c, g, or t
  misc_feature 8: note = n is a, c, g, or t
  misc_feature 14: note = n is a, c, g, or t
  misc_feature 16..18: note = n is a, c, g, or t
  source 1..18: mol_type = other DNA
tnsgaawncgaaantnnn

SEQ ID NO: 9-12 - skipped (4 entries)

SEQ ID NO: 13 - DNA, 11 bp, synthetic construct
  misc_feature 1..11: note = Transcription factor binding site
  misc_feature 11: note = n is a, c, g, or t
  source 1..11: mol_type = other DNA
gcccaygygsn

SEQ ID NO: 14-17 - skipped (4 entries)

SEQ ID NO: 18 - DNA, 14 bp, synthetic construct
  misc_feature 1..14: note = Transcription factor binding site
  misc_feature 10: note = n is a, c, g, or t
  misc_feature 13..14: note = n is a, c, g, or t
  source 1..14: mol_type = other DNA
gycgcgcarngcnn

SEQ ID NO: 19-20 - skipped (2 entries)

SEQ ID NO: 21 - DNA, 12 bp, synthetic construct
  misc_feature 1..12: note = Transcription factor binding site
  source 1..12: mol_type = other DNA
kaccacgtgsyy

SEQ ID NO: 22-23 - skipped (2 entries)

SEQ ID NO: 24 - DNA, 14 bp, synthetic construct
  misc_feature 1..14: note = Transcription factor binding site
  misc_feature 1..2: note = n is a, c, g, or t
  misc_feature 13..14: note = n is a, c, g, or t
  source 1..14: mol_type = other DNA
nnggggcgggggnn

SEQ ID NO: 25 - skipped

SEQ ID NO: 26 - DNA, 13 bp, synthetic construct
  misc_feature 1..13: note = Transcription factor binding site
  misc_feature 1: note = n is a, c, g, or t
  misc_feature 13: note = n is a, c, g, or t
  source 1..13: mol_type = other DNA
ngggggcggggyn

SEQ ID NO: 27-28 - skipped (2 entries)